(19) United States
(12) Patent Application Publication
(10) Pub. No.: US 2024/0423611 A1
(43) Pub. Date: Dec. 26, 2024

(54) SURGICAL SUTURE

(71) Applicant: W. L. Gore & Associates, Inc., Newark, DE (US)

(72) Inventors: Gopalan V. Balaji, Kennett Square, PA (US); Franklin C. Wetherell, Flagstaff, AZ (US)

(21) Appl. No.: 18/748,916

(22) Filed: Jun. 20, 2024

Related U.S. Application Data
(60) Provisional application No. 63/522,278, filed on Jun. 21, 2023.
(60) Provisional application No. 63/661,803, filed on Jun. 19, 2024.

(51) Int. Cl. A61B 17/06

(52) U.S. Cl. CPC A61B 17/06166

(57) ABSTRACT

A surgical suture of a synthetic fiber is provided. The suture includes a non-circular cross-section, and may further include maximal and minimal cross-sectional dimensions with the minimal cross-sectional dimension. The suture may further include a compressible width that supports knot formation and retention, and planar side surfaces that guide knot formation and curvatures. The suture may also include a series of microfilaments that define larger monofilaments or formations that are twisted or braided along the length of the suture.

### CROSS REFERENCE TO RELATED APPLICATION

The present application claims the benefit of U.S. Application No. 63/522,278, filed Jun. 21, 2023, and U.S. Application No. 63/661,803, filed Jun. 19, 2024, which are incorporated herein by reference in their entireties for all purposes.

### FIELD

The present disclosure relates generally to sutures and, more specifically, to nonabsorbable surgical sutures.

### BACKGROUND

Sutures are often used to engage anatomical structures and, in particular, used in the approximation and/or ligation of soft tissue. Sutures typically consist of a filamentous suture thread that may be attached to a needle with a sharp point. Suture threads can be multifilament or monofilament and be made from a variety of materials including absorbable materials that can be absorbed into the body over time and nonabsorbable materials that resist absorption into the body.

It is generally recognized that the United States Pharmacopeia National Formulary (USP-NF), published by the United States Pharmacopeial Convention (Rockville, Maryland, USA), provides authoritative monographs, testing requirements, and other information regarding suture types, properties, and performance requirements. In particular, the USP-NF provides monographs and testing requirements for suture products at the following USP-NF sections:
USP-NF section entitled “Absorbable Surgical Suture”, USP-NF DocID: USPNF_M80190_04_01, https://doi.org/10.31003/USPNF_M80190_04_01;
USP-NF section entitled “Nonabsorbable Surgical Suture”, USP-NF DocID: USPNF_M80200_04_01, https://doi.org/10.31003/USPNF_M80200_04_01;
USP-NF section 861 entitled “Sutures-Diameter”, USP-NF DocID: USPNF_M99650_02_01, https://doi.org/10.31003/USPNF_M99650_02_01; and
USP-NF section 881 entitled “Tensile Strength”, USP-NF DocID: USPNF_M99670_02_01, https://doi.org/10.31003/USPNF_M99670_02_01.
   Each of these USP-NF sections are incorporated by reference herein in their entireties. The USP-NF monographs and testing standards for absorbable and nonabsorbable sutures are recognized as authoritative and applicable to suture products regulated by the U.S. Food and Drug Administration (FDA) in at least the following government publications:
“Surgical Sutures-Performance Criteria for Safety and Performance Based Pathway”, FDA, issued Apr. 11, 2022; and
“Surgical Sutures-Class II Special Controls Guidance Document for Industry and FDA Staff”, FDA, issued Jun. 3, 2003.

Each of these FDA publications are incorporated by reference herein in their entireties. FDA also recognizes the above-described USP-NF sections as “Recognized Consensus Standards” that are used by FDA to facilitate the agency's premarket review process that is required for entry of new suture products into regulated markets.

The USP-NF “Nonabsorbable Surgical Suture” monograph, referenced above, defines three classes of nonabsorbable surgical suture:
Class I nonabsorbable suture is described as being composed of silk or synthetic fibers of monofilament, twisted, or braided construction where the coating, if any, does not significantly affect thickness (e.g., braided silk, polyester, or nylon; monofilament nylon or polypropylene);
Class II nonabsorbable suture is described as being composed of cotton or linen fibers or coated natural or synthetic fibers where the coating significantly affects thickness but does not contribute significantly to strength (e.g., virgin silk sutures); and
Class III nonabsorbable suture is described as being composed of monofilament or multifilament metal wire.

The same USP-NF monograph also provides a Table 1, entitled “Average Knot-Pull Limits of Various Sizes and Diameters of Sutures” and incorporated by reference herein in its entirety, that defines 22 USP sizes of nonabsorbable surgical suture ranging (thinnest to thickest) from 12-0, 11-0, 10-0, 9-0, 8-0, 7-0, 6-0, 5-0, 4-0, 3-0, 2.5-0, 2-0, 0, 1, 2, “3 and 4”, 5, 6, 7, 8, 9, to 10. Table 1 also provides dimensional and tensile strength requirements for each USP size and USP class of nonabsorbable surgical suture. In particular, Table 1 defines that:
Class I nonabsorbable surgical sutures of USP size 12-0 must have an average diameter of 0.001-0.009 mm, an individual diameter that does not exceed 0.015 mm, and an average knot-pull tensile strength of at least 0.01 N or 0.0010 kgf (the kgf value being calculated from 0.01*0.10197 per footnote c of Table 1);
Class I nonabsorbable surgical sutures of USP size 11-0 must have an average diameter of 0.010-0.019 mm, an individual diameter of 0.005-0.025 mm, and an average knot-pull tensile strength of at least 0.06 N or 0.006 kgf (the kgf value being calculated from 0.06*0.10197 per footnote c of Table 1);
Class I nonabsorbable surgical sutures of USP size 10-0 must have an average diameter of 0.020-0.029 mm, an individual diameter of 0.015-0.035 mm, and an average knot-pull tensile strength of at least 0.186 N or 0.019 kgf (the kgf value being calculated from 0.186*0.10197 per footnote c of Table 1);
Class I nonabsorbable surgical sutures of USP size 9-0 must have an average diameter of 0.030-0.039 mm, an individual diameter of 0.025-0.045 mm, and an average knot-pull tensile strength of at least 0.422 N or 0.043 kgf (the kgf value being calculated from 0.422*0.10197 per footnote c of Table 1); and
Class I nonabsorbable surgical sutures of USP size 8-0 must have an average diameter of 0.040-0.049 mm, an individual diameter of 0.035-0.060 mm, and an average knot-pull tensile strength of at least 0.59 N or 0.06 kgf (the kgf value being calculated from 0.59*0.101972 per footnote c of Table 1).

At the present time, it is believed that physicians have limited options to acquire and use Class I nonabsorbable surgical sutures of USP sizes 12-0, 11-0, 10-0, 9-0, or 8-0 for surgical procedures such as ophthalmic surgeries or microsurgeries. A well-known supplier of Class I nonabsorbable surgical sutures is Johnson & Johnson (of New Brunswick, New Jersey, USA) and its subsidiary Ethicon, Inc. which markets nonabsorbable surgical sutures that appear to conform with USP Class I requirements. The 2019 Ethicon Product Catalog lists four Class I nonabsorbable sutures of USP sizes 11-0, 10-0, 9-0, or 8-0 made of the materials described as follows:
PRONOVA® Poly (Hexafluoropropylene-VDF) Suture. The PRONOVA® suture is described as a monofilament suture offered in USP size 8-0 (but not in 11-0, 10-0, or 9-0) and is further described as a “nonabsorbable, sterile surgical suture made from polymer blend of poly (vinylidene fluoride) and poly (vinylidene fluoride-co-hexafluoropropylene)” that is “indicated for use in general soft tissue approximation and/or ligation, including use in cardiovascular and neurological procedures”.
ETHILON® Nylon Suture. The ETHILON® suture is described as a monofilament suture offered in USP sizes 11-0, 10-0, 9-0, and 8-0 and is further described as a “nonabsorbable, sterile surgical monofilament suture composed of the long-chain aliphatic polymers Nylon 6 and Nylon 6,6” that is “indicated for use in general soft tissue approximation and/or ligation, including use in cardiovascular, ophthalmic, and neurological procedures”.
MERSILENE® Polyester Fiber Suture. The MERSILENE® suture is offered in a braided form for USP sizes equal or larger that USP 6-0, and offered in a monofilament form for USP sizes 11-0 and 10-0 (but not in 9-0 and 8-0). The material used in the MERSILENE® suture is described as Poly(ethylene terephthalate) with “high molecular weight, long-chain, linear polyesters having recurrent aromatic rings as an integral component” and is “indicated for use in general soft tissue approximation and/or ligation, including use in cardiovascular, ophthalmic, and neurological procedures”.
PROLENE® Polypropylene Suture. The PROLENE® suture is described as a monofilament suture offered in sizes 10-0, 9-0, and 8-0 (but not in 11-0) and is described as being made of polypropylene and as being “indicated for use in general soft tissue approximation and/or ligation, including use in cardiovascular, ophthalmic and neurological procedures”.

The polymeric materials described in the Ethicon catalog are commonly referred to as VDF (for PRONOVA®), nylon (for ETHILON®), polyester (for MERSILENE®), and polypropylene (for PROLENER). With regard to Class I nonabsorbable sutures of USP size 12-0, a supplier of Class I nonabsorbable sutures is Kono Seisakusho Co. Ltd. (of Chiba, Japan) which markets a 12-0 monofilament nylon suture product under the tradename Crownjun® for microsurgery purposes. As noted above, all of these commercially-available suture products are limited to a monofilament configuration for sizes 12-0, 11-0, 10-0, 9-0, and 8-0, and each product is shown to have a circular cross-section of the monofilament.

It is believed that at least three performance limitations are experienced with existing nonabsorbable surgical suture products of USP sizes 12-0, 11-0, 10-0, 9-0, and 8-0. The first is an undesirable degradation of some suture materials due to hydrolysis. Nylon, for example, has been reported to degrade significantly (for example, over a span of about 1-2 years) even though sutures made with nylon are classified as nonabsorbable. At least one nylon suture manufacturer acknowledges this problem in its labeling where it states that “progressive hydrolysis may cause a gradual loss of up to 20% in its tensile strength each year”. It is also believed that polypropylene sutures may experience hydrolytic degradation over a span of about 5 years and may be significantly compromised over a period of 7 to 14 years or an average of 10 years in certain bioactive environments.

The second performance limitation is an undesirable degradation of tensile strength and material cohesion where a knot is formed with a monofilament suture. It is believed that a knotted monofilament suture subjected to a tensile force may deform the monofilament significantly and reduce suture tensile strength over time, especially in regions of the suture that experience the greatest curvature or greatest material distortion in the knot formation. It is also believed that a knotted monofilament suture subjected to a tensile force experiences tensile forces unevenly across the cross-section of the knotted suture, creating high-stress areas of the cross-section that contribute to suture breakage or material degradation over time. It is further believed that a monofilament surgical suture of USP sizes 12-0, 11-0, 10-0, 9-0, and 8-0 experiences significant deformation when the monofilament is forced to turn sharply to form a knot and, in particular, experiences additional and localized deformative forces where the suture enters and exits a knot. Deformations seen at least on the surface of a monofilament suture where the suture enters or exits a knot are believed to create stress points where the knotted suture is likely to break when subjected to tensile loads and/or to create locations where the distorted material is degraded gradually over time. It is further believed that, when a monofilament suture is subjected to curvatures that create localized stresses and deformations, the monofilament is unable to effectively distribute tensile loads across the cross-section of the monofilament as seen with, for example, braided sutures where tensile-loaded fibers stretch to share the load across the cross-section.

A third performance limitation is inadequate knot retention. It is believed that monofilament sutures maintain a nearly consistent cross-sectional area as the suture turns in the formation of a knot which allows the monofilament to slide axially through the tightened knot over time and subsequently loosen the knot as there is little or no cross-sectional deformation of the monofilament to form surfaces that oppose the axial slippage. It is further believed that a portion of the monofilament suture passing through the knot is not sufficiently compressed and deformed to be pinched and held in place by adjacent monofilament suture portions that are uncompressed or that present a wider cross-sectional area. It is yet further believed that the maintenance of a consistent cross-sectional area or the maintenance of a nearly circular cross-sectional area as the monofilament suture travels through a knot permits axial slipping or untying of the knot over time, as the axial movement of the monofilament is not significantly inhibited by effective dimensional changes in the cross-sectional profile of the monofilament where the monofilament suture is disposed within the knot or at the entry and exit points of the knot.

In view of these and other deficiencies in the present offerings for Class I nonabsorbable surgical sutures of USP sizes 12-0, 11-0, 10-0, 9-0, or 8-0, there is a need for a Class I nonabsorbable surgical suture that meets USP requirements for strength and dimensions, resists material degradation over time, and maintains knot security over time.

### SUMMARY

Surgical sutures having certain physical properties are disclosed herein. The physical properties include, but are not limited to, increased porosity, increased compliance and bendability, and/or decreased coefficient of friction as compared to a surgical suture with a diameter that is similar to or equal to a minimal cross-sectional dimension of the surgical sutures as disclosed herein.

According to one example (“Example 1”), a United States Pharmacopeia (USP) Class I nonabsorbable surgical suture is disclosed herein, which includes a synthetic fiber having an axial length suitable for tissue ligation or approximation. The synthetic fiber has a non-circular cross-section maintained along at least a portion of the axial length of the synthetic fiber.

According to one example (“Example 2”), a United States Pharmacopeia (USP) Class I nonabsorbable surgical suture is disclosed herein, which includes a synthetic fiber having an axial length suitable for tissue ligation or approximation and a cross-section disposed orthogonally to the length at a portion of the axial length. The cross-section includes densified areas surrounding openings disposed between the densified areas. The openings are collapsible when the synthetic fiber is externally compressed in a plane of the cross-section. The USP Class I nonabsorbable surgical suture is sized to conform with at least one of USP size 12-0, USP size 11-0, USP size 10-0, USP size 9-0, and USP size 8-0, and optionally USP size 7-0 and USP size 6-0.

According to one example (“Example 3”), a United States Pharmacopeia (USP) Class I nonabsorbable surgical suture is disclosed herein, which includes a synthetic fiber defining a fiber axis and having an axial length suitable for tissue ligation or approximation and side surfaces each extending axially along at least a portion of the axial length of the synthetic fiber. The axially-extending side surfaces include a flat surface disposed between two curved surfaces. Each of the two curved surfaces curves about the axis from 90 degrees to 180 degrees.

According to one example (“Example 4”), a United States Pharmacopeia (USP) Class I nonabsorbable surgical suture is disclosed herein, which includes a synthetic fiber having an axial length suitable for tissue ligation or approximation and a plurality of microfilaments each having microfilament ends that engage each other end-to-end along a portion of the axial length of the synthetic fiber. The USP Class I nonabsorbable surgical suture is sized to conform with at least one of USP size 12-0, USP size 11-0, USP size 10-0, USP size 9-0, and USP size 8-0, and optionally USP size 7-0 and USP size 6-0.

According to one example (“Example 5”), a United States Pharmacopeia (USP) Class I nonabsorbable surgical suture is disclosed herein, which includes a synthetic fiber having an axial length suitable for tissue ligation or approximation and a cross-section disposed orthogonally to the length at a portion of the axial length. The cross-section is deformable from a first state to a second state. The first state presents a first cross-sectional dimension that deforms to a lesser second cross-sectional dimension in the second state. The deforming is due to a reduction of openings disposed within the cross-section. The USP Class I nonabsorbable surgical suture is sized to conform with at least one of USP size 12-0, USP size 11-0, USP size 10-0, USP size 9-0, and USP size 8-0, and optionally USP size 7-0 and USP size 6-0.

According to one example (“Example 6”), a United States Pharmacopeia (USP) Class I nonabsorbable surgical suture is disclosed herein, which includes a synthetic fiber defining a fiber axis and having an axial length suitable for tissue ligation or approximation and a cross-section disposed orthogonally to the axis along a portion of the axial length. The cross-section defines a flat surface of the synthetic fiber extending along the portion of the axial length. The synthetic fiber is predisposed to twist about the axis to present the flat surface as an inner surface of a curvature formed when the fiber is deformed to form the curvature.

According to one example (“Example 7”), a United States Pharmacopeia (USP) Class I nonabsorbable surgical suture is disclosed herein, which includes a synthetic fiber having an axial length suitable for tissue ligation or approximation and a variable cross-section disposed along a portion of the axial length, The variable cross-section varies between an uncompressed state presenting a first cross-section defining a first peripheral edge circumscribing the fiber at the first cross-section and a compressed state presenting a second cross-section defining a lesser second peripheral edge circumscribing the fiber at the second cross-section. The compressed state is present in the fiber within a knot formed by the fiber. The uncompressed state is present in the fiber adjacent to the knot.

According to one example (“Example 8”), a United States Pharmacopeia (USP) Class I nonabsorbable surgical suture is disclosed herein, which includes a synthetic fiber having an axial length suitable for tissue ligation or approximation and a variable cross-section disposed along a portion of the axial length. The variable cross-section varies between an uncompressed state presenting a first cross-section having first openings disposed within the fiber at the first cross-section and a compressed state presenting a second cross-section having second openings disposed within the fiber at the second cross-section. The second openings have a reduced size compared to the first openings. The compressed state is present in the fiber within a knot formed by the fiber. The uncompressed state is present in the fiber adjacent to the knot. The USP Class I nonabsorbable surgical suture is sized to conform with at least one of USP size 12-0, USP size 11-0, USP size 10-0, USP size 9-0, and USP size 8-0, and optionally USP size 7-0 and USP size 6-0.

According to another example (“Example 9”) further to any one of Examples 1 and 3-8, the synthetic fiber has a cross-section disposed orthogonally to the length at a portion of the axial length. The cross-section includes densified areas surrounding openings disposed between the densified areas. The openings are collapsible when the synthetic fiber is externally compressed in a plane of the cross-section.

According to another example (“Example 10”) further to any one of Examples 1, 2, and 4-8, the synthetic fiber has side surfaces each extending axially along at least a portion of the axial length of the synthetic fiber. The axially-extending side surfaces include a flat surface disposed between two curved surfaces. Each of the two curved surfaces curves about the axis from 90 degrees to 180 degrees.

According to another example (“Example 11”) further to any one of Examples 1-3 and 5-8, the synthetic fiber has a plurality of microfilaments each having microfilament ends that engage each other end-to-end along a portion of the axial length of the synthetic fiber.

According to another example (“Example 12”) further to any one of Examples 1-4 and 6-8, the synthetic fiber has a cross-section disposed orthogonally to the length at a portion of the axial length. The cross-section is deformable from a first state to a second state. The first state presents a first cross-sectional dimension that deforms to a lesser second cross-sectional dimension in the second state. The deforming is due to a reduction of openings disposed within the cross-section.

According to another example (“Example 13”) further to any one of Examples 1-5 and 7-8, the synthetic fiber has a cross-section disposed orthogonally to the axis along a portion of the axial length. The cross-section defines a flat surface of the synthetic fiber extending along the portion of the axial length. The synthetic fiber is predisposed to twist about the axis to present the flat surface as an inner surface of a curvature formed when the fiber is deformed to form the curvature.

According to another example (“Example 14”) further to any one of Examples 1-6 and 8, the synthetic fiber has a variable cross-section disposed along a portion of the axial length. The variable cross-section varies between an uncompressed state presenting a first cross-section defining a first peripheral edge circumscribing the fiber at the first cross-section and a compressed state presenting a second cross-section defining a lesser second peripheral edge circumscribing the fiber at the second cross-section. The compressed state is present in the fiber within a knot formed by the fiber. The uncompressed state is present in the fiber adjacent to the knot.

According to another example (“Example 15”) further to any one of Examples 1-7, the synthetic fiber has a variable cross-section disposed along a portion of the axial length. The variable cross-section varies between an uncompressed state presenting a first cross-section having first openings disposed within the fiber at the first cross-section and a compressed state presenting a second cross-section having second openings disposed within the fiber at the second cross-section. The second openings have a reduced size compared to the first openings. The compressed state is present in the fiber within a knot formed by the fiber. The uncompressed state is present in the fiber adjacent to the knot.

According to another example (“Example 16”) further to any one of the preceding Examples, the synthetic fiber includes at least one microfilament extending at least a portion of the axial length.

According to another example (“Example 17”) further to any one of Examples 1-15, the synthetic fiber includes a plurality of microfilaments with each microfilament having opposing microfilament ends. A portion of the plurality of microfilaments is disposed so that opposing microfilament ends engage each other end-to-end to define a monofilament composed of engaged microfilaments extending at least a portion of the axial length.

According to another example (“Example 18”) further to any one of Examples 1-15, the synthetic fiber includes a plurality of microfilaments with each microfilament having opposing microfilament ends. A first portion of the plurality of microfilaments is disposed so that opposing microfilament ends engage each other end-to-end to define a first monofilament composed of engaged microfilaments. A second portion of the plurality of microfilaments is disposed so that opposing microfilament ends engage each other end-to-end to define a second monofilament composed of engaged microfilaments. The first and second monofilaments are twisted about each other for at least a portion of the axial length.

According to another example (“Example 19”) further to any one of Examples 1-15, the synthetic fiber includes a plurality of microfilaments with each microfilament having opposing microfilament ends. A first portion of the plurality of microfilaments is disposed so that opposing microfilament ends engage each other end-to-end to define a first monofilament composed of engaged microfilaments. A second portion of the plurality of microfilaments is disposed so that opposing microfilament ends engage each other end-to-end to define a second monofilament composed of engaged microfilaments. The first and second monofilaments each defines pathways along the axial length to define a braided structure formed by the first and second monofilaments extending along at least a portion of the axial length.

According to another example (“Example 20”) further to any one of the preceding Examples, the synthetic fiber has a minimal cross-sectional dimension that is measurable as a diameter of the synthetic fiber when the fiber is subjected to a diameter measurement technique set forth in USP Section 861 entitled “Sutures—Diameter”.

According to another example (“Example 21”) further to any one of the preceding Examples, the synthetic fiber has a minimal cross-sectional dimension that is determined by a diameter measurement technique involving the disposition of the fiber between an anvil and a presser foot in a planar arrangement with a compressive force applied to the fiber. The fiber is further subjected to a tension force and is allowed to rotate about the fiber axis to assume an orientation presenting the minimal cross-sectional dimension for measurement as a diameter measurement.

According to another example (“Example 22”) further to any one of the preceding Examples, the synthetic fiber has a minimal cross-sectional diameter that is determined from an average of three measurements taken along the axial length of the fiber.

According to another example (“Example 23”) further to any one of the preceding Examples, the synthetic fiber has a minimal cross-sectional diameter that is determined from an average of three measurements taken along the axial length of the fiber. A USP diameter of the surgical suture is further determined by averaging the three measurements with a plurality of additional similar measurements obtained from at least nine additional samples of the surgical suture.

According to another example (“Example 24”) further to any one of Examples 1, 3, 6, and 7, the USP Class I nonabsorbable surgical suture is sized to conform with at least one of USP size 12-0, USP size 11-0, USP size 10-0, USP size 9-0, and USP size 8-0, and optionally USP size 7-0 and USP size 6-0.

According to another example (“Example 25”) further to Example 24, the synthetic fiber has a minimal cross-sectional diameter that is determined from at least an average of three measurements taken along the axial length of the fiber. The minimal cross-sectional diameter is from 0.001 to and 0.009 mm for USP size 12-0, the minimal cross-sectional diameter is from 0.010 mm to 0.019 mm for USP size 11-0, the minimal cross-sectional diameter is from 0.020 mm to 0.029 mm for USP size 10-0, the minimal cross-sectional diameter is from 0.030 mm to 0.039 mm for USP size 9-0, and the minimal cross-sectional diameter is from 0.040 mm to 0.049 mm for USP size 8-0.

According to another example (“Example 26”) further to any one of the preceding Examples, the synthetic fiber has an average tensile strength when the fiber is subjected to a tensile strength measurement technique set forth in USP Section 881 entitled “Tensile Strength”.

According to another example (“Example 27”) further to any one of the preceding Examples, the synthetic fiber has a tensile strength that is determined by a tensile strength measurement technique involving the disposition of the fiber between two clamps with at least one of the two clamps moving away from the other to apply a measurable tensile force to the suture.

According to another example (“Example 28”) further to any one of the preceding Examples, the synthetic fiber is disposed to form a simple knot along a portion of the axial length prior to being subjected to a tensile strength measurement technique. The simple knot forms a loop through which a portion of the fiber passes through the loop to form the simple knot when respective ends of the fiber are pulled away from each other.

According to another example (“Example 29”) further to any one of the preceding Examples, the synthetic fiber has a tensile strength that is determined from a tensile strength measurement technique. A USP tensile strength of the surgical suture is further determined by averaging the fiber tensile strength with a plurality of additional similar measurements obtained from at least nine additional samples of the surgical suture.

According to another example (“Example 30”) further to Example 24, the synthetic fiber has a fiber tensile strength that is determined with a tensile strength measurement technique, the surgical suture having a USP tensile strength value determined from the fiber tensile strength averaged with additional similar fiber tensile strengths obtained from at least nine additional samples of the surgical suture. The USP tensile strength value is a straight-pull tensile strength of at least 0.01 N for USP size 12-0, the USP tensile strength value is a straight-pull tensile strength of at least 0.06 N for USP size 11-0, the USP tensile strength value is a straight-pull tensile strength of at least 0.186 N for USP size 10-0, the USP tensile strength value is a straight-pull tensile strength of at least 0.422 N for USP size 9-0, and the USP tensile strength value is a knot-pull tensile strength of at least 0.59 N for USP size 8-0.

According to another example (“Example 31”) further to any one of the preceding Examples, the surgical suture has a plurality of varying cross-sectional dimensions including a minimal cross-sectional dimension and a maximal cross-sectional dimension. The minimal and maximal cross-sectional dimensions differ from each other in a range from 1% to 30%.

According to another example (“Example 32”) further to any one of Examples 1-30, the surgical suture has a plurality of varying cross-sectional dimensions including a minimal cross-sectional dimension and a maximal cross-sectional dimension. The minimal and maximal cross-sectional dimensions differ from each other from 1% to 20%.

According to another example (“Example 33”) further to any one of Examples 1-30, the surgical suture has a plurality of varying cross-sectional dimensions including a minimal cross-sectional dimension and a maximal cross-sectional dimension. The minimal and maximal cross-sectional dimensions differ from each other from 1% to 10%.

According to another example (“Example 34”) further to any one of Examples 1-30, the surgical suture has a plurality of varying cross-sectional dimensions including a minimal cross-sectional dimension and a maximal cross-sectional dimension. The minimal and maximal cross-sectional dimensions differ from each other from 2% to 5%.

According to another example (“Example 35”) further to any one of the preceding Examples, the surgical suture has a maximal cross-sectional dimension and a minimal cross-sectional dimension that is less than the maximal cross-sectional dimension. The minimal cross-sectional dimension is from 10 μm to 100 μm, and optionally from 5 μm to 100 μm.

According to another example (“Example 36”) further to any one of the preceding Examples, the surgical suture comprises expanded polytetrafluoroethylene (ePTFE).

According to another example (“Example 37”) further to any one of the preceding Examples, the surgical suture has a maximal cross-sectional dimension and a minimal cross-sectional dimension. The maximal cross-sectional dimension is at least 20% longer than the minimal cross-sectional dimension.

According to another example (“Example 38”) further to any one of the preceding Examples, the surgical suture includes a material that is microporous.

According to another example (“Example 39”) further to any one of the preceding Examples, the surgical suture includes a material with a porosity of from 35% and 55%.

According to another example (“Example 40”) further to any one of the preceding Examples, the surgical suture is colored using a pigment.

According to another example (“Example 41”) further to any one of the preceding Examples, the surgical suture has a matrix tensile strength (MTS) of at least 70 kgf/mm².

According to another example (“Example 42”) further to any one of the preceding Examples, the surgical suture is compliant and bendable to a bending radius of from 0.19 mm to 0.50 mm when two ends of the suture are held under tension by a pair of weights totaling about 100 mg in mass.

According to another example (“Example 43”) further to any one of the preceding Examples, the surgical suture has a knot size of from 50 μm to 100 μm, and optionally from 25 μm to 100 μm.

According to another example (“Example 44”) further to any one of the preceding Examples, the surgical suture has a coefficient of friction equal to or less than 0.02.

According to another example (“Example 45”) further to any one of the preceding Examples, the surgical suture is coated with a hydrophilic coating.

According to another example (“Example 46”) further to any one of the preceding Examples, the surgical suture has a porosity of from 35% to 55% and a minimal cross-sectional dimension from 10 μm to 100 μm, and optionally from 5 μm to 100 μm.

According to another example (“Example 47”) further to any one of the preceding Examples, the surgical suture is labeled as USP size 12-0.

According to another example (“Example 48”) further to any one of Examples 1-46, the surgical suture is labeled as USP size 11-0.

According to another example (“Example 49”) further to any one of Examples 1-46, the surgical suture is labeled as USP size 10-0.

According to another example (“Example 50”) further to any one of Examples 1-46, the surgical suture is labeled as USP size 9-0.

According to another example (“Example 51”) further to any one of Examples 1-46, the surgical suture is labeled as USP size 8-0.

According to another example (“Example 52”) further to any one of Examples 1-46, the surgical suture has a maximal cross-sectional dimension. The maximal cross-sectional dimensions are measured with a noncontact method.

According to another example (“Example 53”) further to Example 52, the noncontact method includes a laser micrometer.

According to another example (“Example 54”) further to Example 52 or 53, the noncontact method is that set forth in USP Section 861 entitled “Sutures-Diameter”.

According to another example (“Example 55”) further to any one of Example 52-54, the noncontact method includes SEM photography.

According to another example (“Example 56”) further to any one of the preceding Examples, SEM photography is used to determine at least one of a USP size, a diameter, a minimal cross-sectional diameter, a maximal cross-sectional diameter, a varying diameter, a knot dimension, a curvature, and a porosity.

According to another example (“Example 57”), a surgical suture kit is disclosed including the surgical suture of any one of the preceding Examples. The surgical suture kit further includes a label accompanying the surgical suture. The label states that the accompanying surgical suture is at least one of USP size 12-0, USP size 11-0, USP size 10-0, USP size 9-0, and USP size 8-0, and optionally USP size 7-0 and USP size 6-0.

According to another example (“Example 58”), a surgical suture includes a synthetic fiber having an axial length suitable for tissue ligation or approximation, the synthetic fiber further defining a cross-section along at least a portion of an axial length of the synthetic fiber having a major dimension and a minor dimension, the major dimension being greater than the minor dimension.

According to another example (“Example 59”) further to Example 58, the cross-section is non-circular, and optionally wherein the cross-section is an ovular cross-section.

According to another example (“Example 60”) further to Example 58, the cross-section is defined along a majority of the axial length of the synthetic fiber.

According to another example (“Example 61”) further to Example 58, the cross-section has a perimeter that includes two or more curvatures that are different from one another, and optionally wherein the plurality of different curvatures differ by at least 20%, 50%, 100%, 200%, 300%, 400%, or 500%.

According to another example (“Example 62”) further to Example 58, the major dimension and the minor dimension of the cross-section differ by at least 20%, 50%, 100%, 200%, 300%, 400%, or 500%.

According to another example (“Example 63”) further to Example 58, the cross-section has a perimeter including a first curved surface and a second curved surface, wherein the first curved surface defines a first center of curvature, and the second curved surface defines a second center of curvature different from the first center of curvature.

According to another example (“Example 64”) further to Example 63, the first and second centers of curvature are different from a centroid of the cross-section.

According to another example (“Example 65”) further to Example 58, the cross-section has a perimeter defined by a porous surface, and, optionally wherein the porous surface is characterized by a plurality of fibers of material separated by spaces.

According to another example (“Example 66”) further to Example 58, the surgical suture has a periphery comprising a flat side extending at least partially along the axial length of the synthetic fiber.

According to another example (“Example 67”) further to Example 58, the cross-section of the surgical suture defines a periphery having a first end and a second end defining the major dimension therebetween and a first side and a second side defining the minor dimension therebetween, and further wherein at least one of the first, second, third, and fourth sides is relatively flatter than another one of the first, second, third, and fourth sides, and optionally wherein at least one of the first, second, third, and fourth sides is substantially flat.

According to another example (“Example 68”) further to Example 58, the cross-section is included in a bent portion of the suture that defines an radius of curvature when placed in a bent orientation, and further wherein the suture self-orients with a minor axis defined along the minor dimension positioned more parallel to the radius of curvature than a major axis defined along the major dimension, and further wherein the major axis is oriented more orthogonal to the radius of curvature than the minor axis.

According to another example (“Example 69”) further to Example 68, the bent portion of the surgical suture is part of a knot with a maximum cross-sectional dimension of from 50 μm to 100 μm, and optionally from 25 μm to 100 μm.

According to another example (“Example 70”) further to Example 58, the cross-section of the suture defines an inner face and an outer face opposite the inner face, the minor dimension extending between the inner and outer faces, and further wherein the suture has an inner-facing bias along the axial length in response to the suture being bent.

According to another example (“Example 71”) further to any one of Examples 58-70, the suture is a United States Pharmacopeia (USP) Class I nonabsorbable surgical suture.

According to another example (“Example 72”), a suture meets United States Pharmacopeia (USP) Class I Size 4-0 or smaller nonabsorbable surgical suture requirements. The suture includes a synthetic fiber having an axial length suitable for tissue ligation or approximation and a transverse cross-section including densified areas surrounding openings disposed between the densified areas, the openings being collapsible when the synthetic fiber is transversely compressed.

According to another example (“Example 73”) further to Example 72, the surgical suture is compliant and bendable to a bending radius of approximately 0.19 mm when two ends of the suture are held under tension by a pair of weights totaling approximately 100 mg in mass.

According to another example (“Example 74”), a suture meets United States Pharmacopeia (USP) Class I Size 4-0 or smaller nonabsorbable surgical suture requirements. The suture includes a synthetic fiber having an axial length suitable for tissue ligation or approximation and a variable cross-section defined along a portion of the axial length, a first, unknotted portion of the axial length having an uncompressed state in which the first, unknotted portion presents a first cross-section having a first peripheral edge circumscribing the fiber and a second, knotted portion of the axial length having a compressed state in which the second, knotted portion presents a second cross-section having a second peripheral edge circumscribing the fiber, the second peripheral edge having a greater length than the first peripheral edge, wherein the first, unknotted portion of the axial length is located adjacent the second, knotted portion.

According to another example (“Example 75”), a suture meets United States Pharmacopeia (USP) Class I Size 4-0 or smaller nonabsorbable surgical suture requirements. The suture includes a synthetic fiber having an axial length suitable for tissue ligation or approximation and a variable cross-section disposed along a portion of the axial length, the variable cross-section varying between an uncompressed state presenting a first cross-section having first openings disposed within the fiber at the first cross-section and a compressed state presenting a second cross-section having second openings disposed within the fiber at the second cross-section, the second openings having a reduced size compared to the first openings, the compressed state being present in the fiber within a knot formed by the fiber, the uncompressed state being present in the fiber adjacent to the knot.

According to another example (“Example 76”) further to any one of Examples 72-75, the suture is sized to conform with at least one of USP size 12-0, USP size 11-0, USP size 10-0, USP size 9-0, and USP size 8-0, and optionally USP size 7-0 and USP size 6-0.

According to another example (“Example 77”), a suture includes a fiber formed of a synthetic, porous material, the fiber having a compressible, non-circular cross-section and defining a fiber axis and having an axial length suitable for tissue ligation or approximation and side surfaces each extending axially along at least a portion of the axial length of the synthetic fiber, the fiber being in a knotted configuration such that the fiber has a first portion defining a first porosity and a second portion defining a second porosity greater than the first porosity, the first portion corresponding to the knotted portion and the second portion corresponding to an unknotted portion.

According to another example (“Example 78”) further to Example 77, the first porosity is defined by first pore openings, and the second porosity is defined by second pore openings having larger average pore sizes than the first pore openings.

According to another example (“Example 79”) further to Example 77 or 78, the suture is a United States Pharmacopeia (USP) Class I nonabsorbable surgical suture.

According to another example (“Example 80”), a United States Pharmacopeia (USP) Class I nonabsorbable surgical suture includes a synthetic fiber defining a fiber axis and having an axial length suitable for tissue ligation or approximation and side surfaces each extending axially along at least a portion of the axial length of the synthetic fiber, the fiber having pore openings that transition from a first uncompressed configuration in the absence of external forces applied to the fiber to a second compressed configuration in response to the external force being applied to the fiber, the fiber having a minimal cross-sectional dimension and a maximal cross-sectional dimension, wherein at least one of the minimal cross-sectional dimension or the maximal cross-sectional dimension is reduced by at least 30% in response to the fiber transitioning from the first uncompressed configuration to the second compressed configuration.

According to another example (“Example 81”) further to Example 80, the surgical suture is substantially non-biodegradable.

According to another example (“Example 82”) further to Example 80 or 81, the surgical suture has a porosity of from 35% to 55% and a minimal cross-sectional dimension from 5 μm to 100 μm.

According to another example (“Example 83”) further to Example 80-82, the surgical suture is compliant and bendable to a bending radius of from 0.19 mm to 0.50 mm when two ends of the suture are held under tension by a pair of weights totaling about 100 mg in mass.

According to another example (“Example 84”) further to Example 80-83, the surgical suture has a knot size of from 50 μm to 100 μm, and optionally from 25 μm to 100 μm.

According to another example (“Example 85”), a medical device includes a first component and a second component. The medical device also includes the surgical suture of any one of Examples 1-84, where the surgical suture engages with each of the first and second components to secure the first component to the second component.

According to another example (“Example 86”) further to Example 85, the medical device is a heart valve.

According to another example (“Example 87”), a medical device includes the surgical suture of any one of Examples 1-84, and a biocompatible component usable in a body of a patient, wherein the suture is coupled to the biocompatible component.

According to another example (“Example 88”) further to Example 87, the biocompatible component includes at least one of: an implant, a constraining sleeve, a graft, a valve leaflet, or a stent support frame.

According to another example (“Example 89”), a method of securing a first component that is biocompatible and suitable for placement in a body of a patient and a second component that is biocompatible and suitable for placement in a body of a patient, includes securing the first component to the second component using a surgical suture with a non-circular cross-section.

According to another example (“Example 90”) further to Example 89, the surgical suture is characterized by a transverse cross-section including densified areas surrounding openings disposed between the densified areas, and further wherein the method includes forming a knot in the suture such that the synthetic fiber is externally compressed in a plane of the cross-section and the openings are collapsed.

According to another example (“Example 91”) further to Example 89 or 90, the surgical suture is characterized by a transverse cross-section having a maximal cross-sectional dimension and a minimal cross-sectional dimension, the method including forming a knot in the suture, the knot including a plurality of bends in the suture, wherein the suture is biased to orient with the maximal cross-sectional dimension perpendicular to the direction of curvature of at least one of the plurality of bends in the suture.

According to another example (“Example 92”) further to any one of Example 89-91, the surgical suture is a USP Class I nonabsorbable surgical suture sized to conform with at least one of USP size 12-0, USP size 11-0, USP size 10-0, USP size 9-0, and USP size 8-0, and optionally USP size 7-0 and USP size 6-0.

According to another example (“Example 93”) further to any one of Example 89-92, the method is performed as part of a medical device manufacturing process.

According to another example (“Example 94”) further to any one of Example 89-93, the method is performed as part of implanting a medical device in a body of a patient.

According to another example (“Example 95”), a method of treating an eye condition includes using the suture of any one of Examples 1-84 for an ophthalmic ligation or approximation procedure.

According to another example (“Example 96”) further to any one of Examples 1-84, the suture is manufactured and prior to use in a body of a patient.

According to another example (“Example 97”) further to any one of Examples 1-84, the suture is following use in a body of a patient.

The foregoing Examples are just that, and should not be read to limit or otherwise narrow the scope of any of the concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1A is an illustration of a suture with a thread and a needle attached thereto in accordance with an embodiment;

FIG. 1B is an illustration of a cross-sectional view of the thread of the suture in accordance with an embodiment;

FIG. 1C is an illustration of a cross-sectional view of the thread of the suture showing the pore openings therein in accordance with an embodiment;

FIG. 1D is a photograph of a top view of a surgical suture kit or package with a label indicating the size of the suture in accordance with an embodiment;

FIG. 1E is a cross-sectional diagram of the tread of the suture showing the curved surfaces and centers of curvatures thereof in accordance with an embodiment;

FIG. 2 is a scanning electron microscope (SEM) image of a thread of the suture in accordance with an embodiment, the image is to the scale or dimension shown;

FIGS. 2A and 2B are SEM images of a thread of a suture of a prior-art ETHILON® nylon suture and a prior-art ETHILON® polypropylene (PP) suture, respectively, the images are to the scale or dimension shown;

FIG. 3 is an SEM image of a thread of the suture in accordance with an embodiment, the image is to the scale or dimension shown;

FIGS. 3A and 3B are SEM images of a thread of a suture of a prior-art ETHILON® nylon suture and a prior-art ETHILON® PP suture, respectively, the images are to the scale or dimension shown;

FIG. 4 is an SEM image of a thread of the suture in accordance with an embodiment, the image is to the scale or dimension shown;

FIGS. 4A and 4B are SEM images of a thread of a suture of a prior-art ETHILON® nylon suture and a prior-art ETHILON® PP suture, respectively, the images are to the scale or dimension shown;

FIG. 5 is an SEM image of a thread of the suture in accordance with an embodiment, the image is to the scale or dimension shown;

FIGS. 5A and 5B are SEM images of a thread of a suture of a prior-art ETHILON® nylon suture and a prior-art ETHILON® PP suture, respectively, the images are to the scale or dimension shown;

FIG. 6 is an SEM image of a cross-section of a thread of the suture in accordance with an embodiment, the image is to the scale or dimension shown;

FIGS. 6A and 6B are SEM images of a cross-section of the thread of a suture of a prior-art ETHILON® nylon suture and a prior-art ETHILON® PP suture, respectively, the images are to the scale or dimension shown;

FIG. 7 is an SEM image of a cross-section of a thread of the suture in accordance with an embodiment, the image is to the scale or dimension shown;

FIGS. 7A and 7B are SEM images of a cross-section of the thread of a suture of a prior-art ETHILON® nylon suture and a prior-art ETHILON® PP suture, respectively, the images are to the scale or dimension shown;

FIG. 8A is an image of a testing apparatus for testing the bending radius of a suture according to embodiments disclosed herein;

FIG. 8B is an image of a measured portion of the suture to determine the bending radius as captured using the testing apparatus according to embodiments disclosed herein, the image is to the scale or dimension shown;

FIGS. 9A-D are SEM images of the bent portions of the presently disclosed suture according to embodiments disclosed herein, the images are to the scale or dimension shown;

FIGS. 10A and 10B are SEM images of the bent portions of the prior-art nylon suture, the images are to the scale or dimension shown;

FIGS. 10C and 10D are SEM images of the bent portions of the prior-art PP suture, the images are to the scale or dimension shown;

FIG. 11 is a graph comparing the radius vs mass of each suture as shown in FIGS. 9A-D and 10A-D;

FIGS. 12A-C are images showing a testing method for determining a knot size of a suture according to embodiments disclosed herein;

FIG. 13 is an SEM image of a tensioned knot of the suture according to embodiments disclosed herein, the image is to the scale or dimension shown;

FIG. 13A is an SEM image of a tensioned knot of the prior-art ETHILON® nylon suture, the image is to the scale or dimension shown;

FIG. 13B is an SEM image of a tensioned knot of the prior-art ETHILON® PP suture, the image is to the scale or dimension shown;

FIG. 14 is an enlarged view of a portion of FIG. 13 to show certain details according to embodiments disclosed herein;

FIG. 15A is an SEM image of a cross-sectional surface of a prior-art “CV4” suture as manufactured by W. L. Gore and Associates, Inc., the image is to the scale or dimension shown;

FIG. 15B is an enlarged view of a portion of FIG. 15A to show certain details of the suture, the image is to the scale or dimension shown;

FIG. 15C is an SEM image of a tensioned knot of the suture of FIG. 15A, the image is to the scale or dimension shown;

FIG. 15D is an SEM image of a surface of the suture of FIG. 15A, the image is to the scale or dimension shown;

FIG. 15E is an enlarged view of a portion of FIG. 15D to show certain details of the suture, the image is to the scale or dimension shown;

FIG. 15F is an SEM image of a surface of the suture of FIG. 15A, the image is to the scale or dimension shown;

FIG. 15G is an enlarged view of a portion of FIG. 15F to show certain details of the suture, the image is to the scale or dimension shown;

FIG. 15H is an enlarged view of a portion of FIG. 15G to show certain details of the suture, the image is to the scale or dimension shown;

FIG. 16A is an SEM image of a cross-sectional surface of a prior-art “CV6” suture as manufactured by W. L. Gore and Associates, Inc., the image is to the scale or dimension shown;

FIG. 16B is an enlarged view of a portion of FIG. 16A to show certain details of the suture, the image is to the scale or dimension shown;

FIG. 16C is an SEM image of a tensioned knot of the suture of FIG. 16A, the image is to the scale or dimension shown;

FIG. 16D is an SEM image of a surface of the suture of FIG. 16A, the image is to the scale or dimension shown;

FIG. 16E is an SEM image of a surface of another prior-art “CV6” suture as manufactured by W. L. Gore and Associates, Inc., the image is to the scale or dimension shown;

FIG. 16F is an enlarged view of a portion of FIG. 16E to show certain details of the suture, the image is to the scale or dimension shown;

FIG. 16G is an enlarged view of a portion of FIG. 16F to show certain details of the suture, the image is to the scale or dimension shown;

FIG. 17 is a perspective view of a prosthetic heart valve, in accordance with embodiments disclosed herein; and

FIG. 18 is a cross-sectional view of a heart having a prosthetic valve mounted in the mitral valve annulus, in accordance with embodiments disclosed herein.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

The term “fibril” as used herein describes an elongated piece of material such as a polymer, where the length and width are substantially different from each other. For example, a fibril may resemble a piece of string or fiber, where the width (or thickness) is much shorter or smaller than the length.

The term “node” as used herein describes a connection point of at least two fibrils, where the connection may be defined as a location where the two fibrils come into contact with each other, permanently or temporarily. In some examples, a node may also be used to describe a larger volume of polymer than a fibril and where a fibril originates or terminates with no clear continuation of the same fibril through the node. In some examples, a node has a greater width but a smaller length than the fibril.

As used herein, “nodes” and “fibrils” may be used to describe objects that are usually, but not necessarily, connected or interconnected, and have a microscopic size, for example. A “microscopic” object may be defined as an object with at least one dimension (width, length, or height) that is substantially small such that the object or the detail of the object is not visible to the naked eye or difficult, if not impossible, to observe without the aid of a microscope (including but not limited to a scanning electron microscope or SEM, for example) or any suitable type of magnification device.

The term “suture diameter” can be defined by the United States Pharmacopeia (USP) at USP29-NF24, p. 2775 (2020), which sets forth a dead-weight method for determining the diameter of sutures (and incorporated by reference herein in its entirety). It is to be understood that a variety of suture lengths may be used with the sutures described herein, and the term “diameter” is associated with a circular (or substantially circular) periphery. Example definitions for types of sutures, suture sizes, suture strength requirements, and other features of sutures are provided in the United States Pharmacopeia (USP) at: U.S. Department of Health and Human Services, Food and Drug Administration, Center for Devices and Radiological Health. (2022 April 11). “Surgical Sutures—Performance Criteria for Safety and Performance Based Pathway: Guidance for Industry and Food and Drug Administration Staff.” Retrieved Aug. 15, 2022, from https://www.fda.gov/media/157490/download. The document is incorporated herein in its entirety. The USP designation of suture size runs from 0 to 7 in the larger range and 1-0 to 12-0 in the smaller range; in the smaller range, the higher the value preceding the hyphenated zero, the smaller the suture diameter. The actual diameter of a suture will depend on the suture material, so that, by way of example, a suture of size 5-0 and made of collagen will have a diameter of 0.15 mm, while sutures having the same USP size designation but made of a synthetic absorbable material or a non-absorbable material will each have a diameter of 0.1 mm. The selection of suture size for a particular purpose depends upon factors such as the nature of the tissue to be sutured and the importance of cosmetic concerns; while smaller sutures may be more easily manipulated through tight surgical sites and are associated with less scarring, the tensile strength of a suture manufactured from a given material tends to decrease with decreasing size. It is to be understood that the sutures and methods of manufacturing sutures disclosed herein are suited to a variety of diameters, including without limitation 7, 6, 5, 4, 3, 2, 1, 0, 1-0, 2-0, 3-0, 4-0, 5-0, 6-0, 7-0, 8-0, 9-0, 10-0, 11-0, and 12-0. For example, modern suture sizes range from USP 5 (heavy braided suture for orthopedic procedures) to USP 11-0 (fine monofilament suture for ophthalmic procedures). Table 1 below shows an example of the upper and lower limit on the average diameter for a surgical suture for each USP size and its metric size (gauge number) equivalent.

TABLE 1
USP size and metric size comparison for thread sizes

| | Metric | Minimum | Maximum |
|---|---|---|---|
| USP | size | average | average |
| size | (gauge no.) | diameter (mm) | diameter (mm) |
| 12-0 | 0.01 | 0.001 | 0.009 |
| 11-0 | 0.1 | 0.010 | 0.019 |
| 10-0 | 0.2 | 0.020 | 0.029 |
| 9-0 | 0.3 | 0.030 | 0.039 |
| 8-0 | 0.4 | 0.040 | 0.049 |
| 7-0 | 0.5 | 0.050 | 0.069 |
| 6-0 | 0.7 | 0.070 | 0.099 |
| 5-0 | 1.0 | 0.100 | 0.149 |
| 4-0 | 1.5 | 0.150 | 0.199 |
| 3-0 | 2.0 | 0.200 | 0.249 |
| 2-0 | 3.0 | 0.300 | 0.349 |
| 0 | 3.5 | 0.350 | 0.399 |
| 1 | 4.0 | 0.400 | 0.499 |
| 2 | 5.0 | 0.500 | 0.599 |

The term “suture needle” (or simply “needle” as used herein) refers to any suitable needle that is used to deploy sutures into tissue, which may come in many different shapes, forms and compositions. The needle may be a traumatic needle or an atraumatic needle. Traumatic needles have channels or drilled ends (that is, holes or eyes) and are supplied separate from the suture thread and are threaded on site. Atraumatic needles are eyeless and are attached to the suture at the factory by swaging or other methods whereby the suture material is inserted into a channel at the blunt end of the needle which is then deformed to a final shape to hold the suture and needle together. As such, atraumatic needles do not require extra time on site for threading and the suture end at the needle attachment site is generally smaller than the needle body. In the traumatic needle, the thread comes out of the needle's hole on both sides and often the suture rips the tissues to a certain extent as it passes through. Most modern sutures are swaged atraumatic needles. Atraumatic needles may be permanently swaged to the suture or may be designed to come off the suture with a sharp straight tug. These “pop-offs” may be used for interrupted sutures, where each suture is only passed once and then tied. For barbed sutures that are uninterrupted, these atraumatic needles may be more preferable in some examples. Suture needles may also be classified according to the geometry of the tip or point of the needle. For example, needles may be (i) “tapered” whereby the needle body is round and tapers smoothly to a point; (ii) “cutting” whereby the needle body is triangular and has a sharpened cutting edge on the inside; (iii) “reverse cutting” whereby the cutting edge is on the outside; (iv) “trocar point” or “taper cut” whereby the needle body is round and tapered, but ends in a small triangular cutting point; (v) “blunt” points for sewing friable tissues; (vi) “side cutting” or “spatula points” whereby the needle is flat on top and bottom with a cutting edge along the front to one side (these are typically used for eye surgery). Suture needles may also be of several shapes including, (i) straight, (ii) half curved or ski, (iii) a fraction (e.g., ¼, ½, etc.) of a circle, or (iv) a compound curve. The sutures described herein may be deployed with a variety of needle types (including without limitation curved, straight, long, short, micro, and so forth), needle cutting surfaces (including without limitation, cutting, tapered, and so forth), and needle attachment techniques (including without limitation, drilled end, crimped, and so forth). Moreover, the sutures described herein may themselves include sufficiently rigid and sharp ends so as to dispense with the requirement for deployment needles altogether.

### Description of Various Embodiments

Knot tying of a suture can cause a range of complications, including, but not limited to: (i) spitting, which is a condition where the suture, usually a knot, pushes through the skin after a subcutaneous closure; (ii) infection, where bacteria are often able to attach and grow in the spaces created by a knot; (iii) bulk/mass, where a significant amount of suture material left in a wound is the portion that comprises the knot; (iv) slippage, in which knots can slip or come untied; and (v) irritation caused by the knots serving as a bulk “foreign body” in a wound. Suture loops associated with knot tying may lead to ischemia, e.g., knots can create tension points that can strangulate tissue and limit blood flow to the region, and increased risk of dehiscence or rupture at the surgical wound. The present disclosure relates to surgical sutures that use an expanded polytetrafluoroethylene (ePTFE) material and capable of reducing, minimizing, or eliminating such aforementioned problems.

In some embodiments, the suture has a minimal cross-sectional dimension of from 10 μm to 100 μm, or from 5 μm to 100 μm. The suture may have a maximal cross-sectional dimension of greater than about 100 μm. The maximal cross-sectional dimension may be at least 20% longer than the minimal cross-sectional dimension. The minimal cross-sectional dimension may be 80% or less than the maximal cross-sectional dimension. The suture may have an effective cross-sectional diameter of from 10 μm to 100 μm, or from 5 μm to 100 μm. The ePTFE material may be porous or microporous. The porosity of the material may be from 35% to 55%.

In some examples, the porosity may be determined using average diameter and average mass of the material, in which case the ePTFE material that is used in the suture may have a diameter of approximately 25 μm and an average mass of approximately 1.5 mg. The suture may be colored using a pigment, or a plurality of pigment particles stored or enclosed within the pores of the ePTFE material. The suture in some examples may have a matrix tensile strength (MTS) of at least about 100 Ksi (or approximately 70.3 kgf/mm²), or the suture in some examples may have the MTS of no greater than about 160 Ksi (or approximately 112.5 kgf/mm²), or any other suitable value or range therebetween. This range meets the USP standard for either a 10-0 suture (0.025 kgf on average) or a 9-0 suture (0.050 kgf on average) as known in the art.

In some examples, the suture may be compliant and bendable to a curvature radius (or bending radius), when held under tension using weights of about 100 mg using the testing method as explained further herein, of from 0.19 mm to 0.22 mm, from 0.22 mm to 0.25 mm, from 0.25 mm to 0.30 mm, from 0.30 mm to 0.32 mm, from 0.32 mm to 0.35 mm, from 0.35 mm to 0.37 mm, from 0.37 mm to 0.40 mm, from 0.40 mm to 0.45 mm, from 0.45 mm to 0.50 mm, or any suitable value or range therebetween, or any suitable combination of ranges thereof. In some examples, the suture may have a knot size of from 25 μm to 50 μm, from 50 μm to 60 μm, from 60 μm to 70 μm, from 70 μm to 80 μm, from 80 μm to 90 μm, from 90 μm to 100 μm, or any suitable value or range therebetween, or any suitable combination of ranges thereof. The suture may have a coefficient of friction that is less than about 0.02. The suture may be coated with a hydrophobic coating.

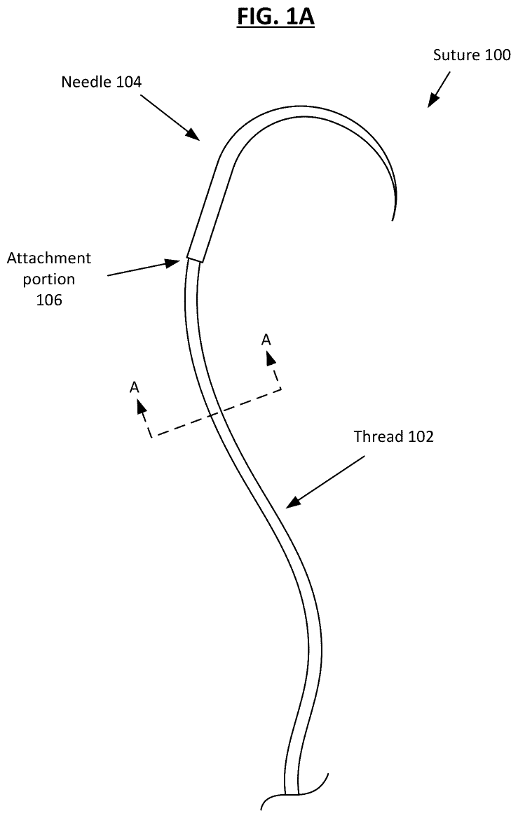

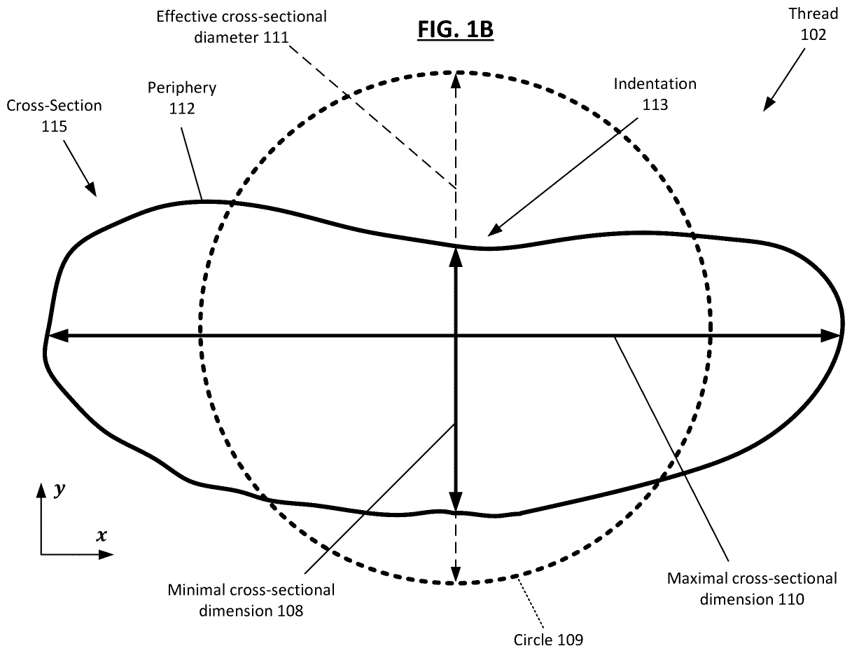

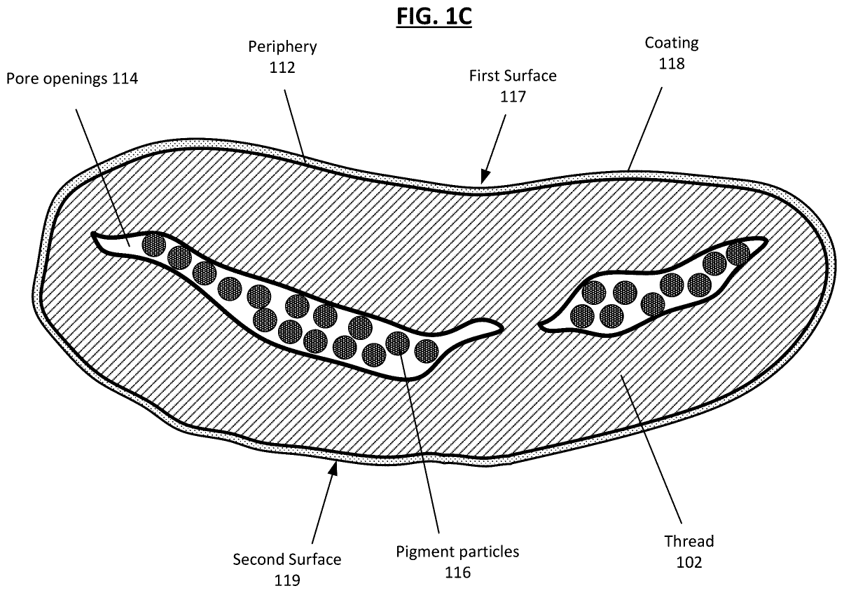

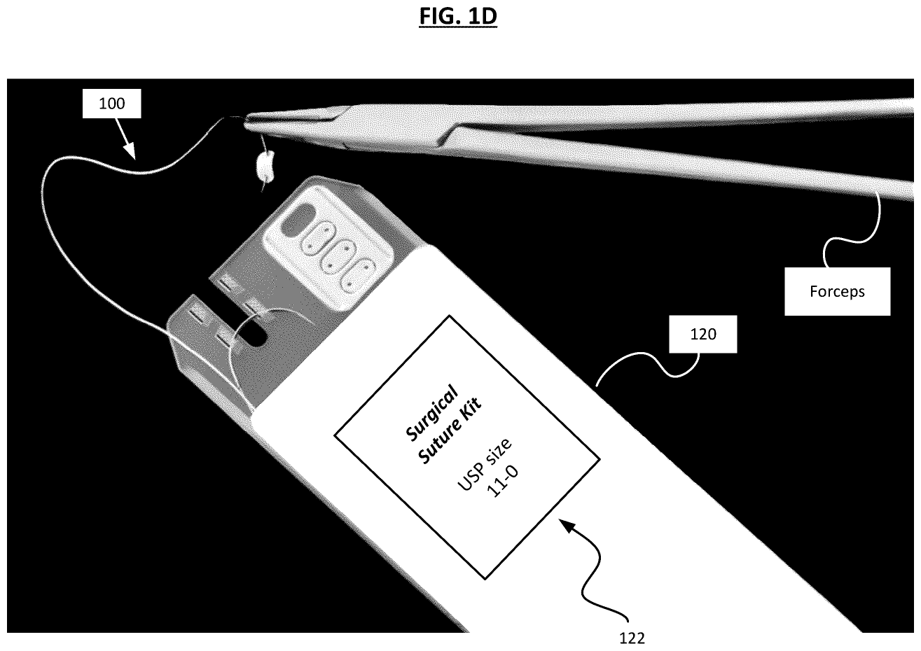

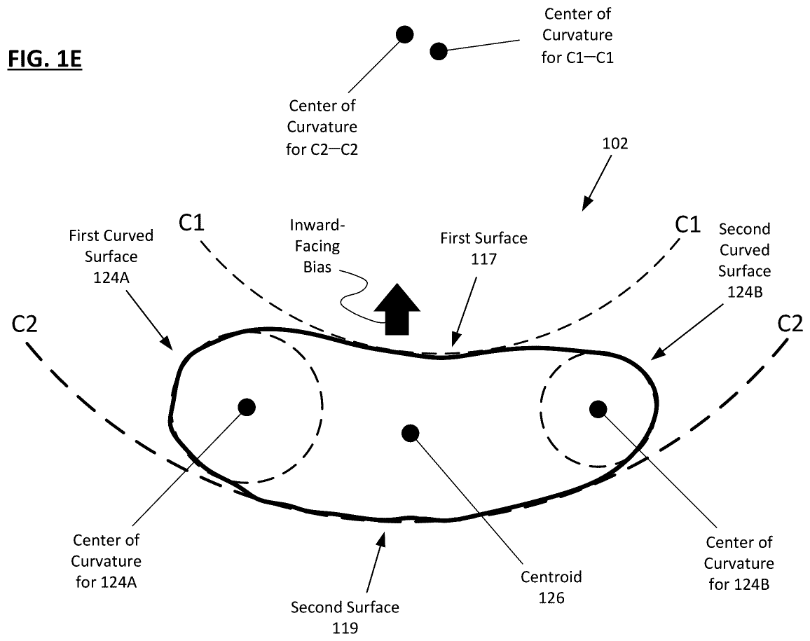

FIG. 1A illustrates a suture 100 having a thread 102 (which may also be referred to herein as a cord, a fiber, or filaments, for example) and a needle 104 (which may also be referred to as a tissue-piercing or tissue-cutting feature) attached to the thread at an attachment portion 106, for example by inserting an end portion of the thread 102 into a needle hole of the needle 104 such that the end portion of the thread 102 fills the needle hole such that the thread material can reduce needle hole leakage and blood loss and also shorten time to hemostasis after operation. In some examples, the thread 102 may be releasably attached to the needle 104. The needle 104 may have a straight or curved (e.g., arched) configuration. The needle 104 may have sufficient sharpness to effectively pierce or cut any suitable tissue such as the tissue of a part of a body of a patient, such as the patient's organ, including but not limited to skin, eye, heart, or other anatomical feature. Needle attachment methods may include crimping, swaging, using adhesives, and so forth. The suture thread may attached to the suture needle using methods such as crimping, swaging and adhesives. The point of attachment of the suture to the needle is known as the swage.

In some examples, the thread 102 of the suture 100 may be made of an expanded polymer such as ePTFE which has a microporous structure made of a plurality of nodes interconnected by fibrils and is nonabsorbable. The thread 102 of the suture 100 may be monofilament or multifilament. The thread 102 may have a porosity of from 35% to 40%, from 40% to 45%, from 45% to 50%, from 50% to 55%, or any other suitable value, range, or combination of ranges therebetween. Porosity is defined as a fraction of void or space within a material, such as those measured using volume and mass of the material. Porosity may also be calculated based on a void fraction determined by dividing the volume of the void by the volume of the material itself.

Benefits of using the suture as disclosed herein include, but are not limited to, reduction in thickness (or minimal cross-sectional dimension) that achieves the same or similar strength (for example, tensile strength of the suture) and durability as compared to a prior-art suture (which may be made of materials such as nylon or polypropylene) having a substantially round cross-section with a greater diameter than the thickness of the suture as disclosed herein. In some examples, the suture has the advantage of being nondegradable (or non-biodegradable) over extended periods as compared with prior-art sutures made of materials such as polypropylene which lasts on average of about 10 years (e.g., about 7 to 14 years) before biodegradation. In some implementations, such as ophthalmic suturing, the suture as disclosed herein provides long-term stability and permanence (e.g., no or very limited degradation over time such as over a span of 5 years, 10 years, 15 years, 20 years, 25 years, 30 years, or any other range therebetween, due to degradation caused by factors including but not limited to hydrolytic or enzymic degradation, or due to other body chemistry) as well as preventing passive or torque-induced tilt, as well as allowing for fine-tuning of centration after posterior chamber intraocular lens (PC IOL) placement, for example.

Furthermore, the porosity or microporosity of the suture may facilitate knot retention so as to prevent any undoing of knots that are formed during procedure, as well as facilitating a smaller knot size than would be otherwise feasible in prior-art sutures with greater diameter, for example. In some examples, the porosity also allows for the suture to be colored using pigments for increased visibility during procedures. The suture as presently disclosed also achieves improved conformability and a tighter curvature radius as compared to prior-art sutures as explained herein (e.g., nylon suture), due to the microporosity. Furthermore, lubricity or wettability of the suture material, such as ePTFE, also facilitates the knots to be repositioned even after the knot throws have been made during procedure. The suture material, such as the ePTFE, also shows superior tensile strength compared to prior-art suture materials including but not limited to nylon. In some examples, the wettability may be increased by applying a hydrophilic coating (e.g., a coating 118 as further explained herein with respect to FIG. 1C) to the suture surface (e.g., a periphery 112, also referred to as a perimeter or an outer perimeter). By wetting the suture surface, the suture material retains the knot as it is tied due to the interfacial force of water.

FIG. 1B illustrates a cross-sectional view of the thread 102 as cut perpendicularly with respect to the length of the thread 102 along the line A-A as shown in FIG. 1A. A periphery 112 of a cross-section 115 of the thread 102 defines a minimal cross-sectional dimension 108 (or minor dimension) and a maximal cross-sectional dimension 110 (or major dimension that is greater by the minor dimension). The minimal dimension is defined as the smallest measurement of thickness (e.g., length measured along the y-axis as viewed from the figure) across the cross-section of the thread 102, which may have a generally noncircular shape as shown, and the maximal dimension is defined as the largest measurement of width (e.g., length measured along the x-axis as viewed from the figure). In some examples, the minimal cross-sectional dimension 108 may be measurable as a diameter of the thread or fiber 102 when the thread or fiber 102 is subjected to a diameter measurement technique set forth in USP Section 861 entitled “Sutures-Diameter.” Cross-sectional dimensions of the suture or thread may be measured with a noncontact method, such as a laser micrometer.

In some examples, the minimal cross-sectional dimension 108 may be determined by a diameter measurement technique involving the disposition of the thread or fiber 102 between an anvil and a presser foot in a planar arrangement with a compressive force applied to the thread or fiber 102, which is further subjected to a tension force to allow the thread or fiber 102 to rotate about the axis to assume an orientation presenting the minimal cross-sectional dimension 108 for measurement as a diameter measurement. In some examples, the minimal cross-sectional diameter 108 may be determined from an average of three measurements taken along the axial length of the thread or fiber 102. In some examples, the minimal cross-sectional diameter 108 may be determined from an average of three measurements taken along the axial length of the thread or fiber 102.

A USP diameter of the surgical suture may further be determined by averaging the three measurements with a plurality of additional similar measurements obtained from at least nine additional samples of the surgical suture. In some examples, the minimal cross-sectional diameter 108 may be determined from at least an average of three measurements taken along the axial length of the thread or fiber 102. Accordingly, the minimal cross-sectional diameter may be from 0.001 mm to 0.009 mm for USP size 12-0, from 0.010 mm to 0.019 mm for USP size 11-0, from 0.020 mm to 0.029 mm for USP size 10-0, from 0.030 mm to 0.039 mm for USP size 9-0, from 0.040 mm to 0.049 mm for USP size 8-0, from 0.050 mm to 0.069 mm for USP size 7-0, and from 0.070 mm to 0.099 mm for USP size 6-0. The minimal/maximal dimensions and/or the cross-sectional shape may be defined along a majority of the axial length of the thread or fiber 102. Additionally, the minimal/maximal dimensions and/or the cross-sectional shape may vary along the axial length of the thread or fiber 102.

The thread 102 may be a fiber, such as a synthetic fiber, having an axial length suitable for tissue ligation or approximation. The fiber may be used for an ophthalmic ligation or approximation, or manufactured to be used in a body of a patient. The fiber has a compressible, non-circular cross-section as illustrated in FIG. 1B that is maintained along at least a portion of the axial length of the synthetic fiber. In some examples, the cross-section of the thread 102 may be deformable from a first state to a second state. The first state presents a first cross-sectional dimension that deforms to a lesser second cross-sectional dimension in the second state. The deformation may be due to a reduction of openings disposed within the cross-section. For example, the difference from the first state to the second state may be defined by a reduction in the minimal cross-sectional dimension 108 and/or a reduction in the maximal cross-sectional dimension 110.

The shape assumed by the cross-section, or the shape of the periphery 112, is non-circular, including but not limited to, ovular, elliptical, polygonal, substantially flattened, oblong, egg-shaped (crooked egg curve), kidney-shaped, heart-shaped (cardioid), or bean-shaped (bean curve), for example. In some examples, the minimal cross-sectional dimension 108 may range between approximately 5 μm and 10 μm, 10 μm and 20 μm, 20 μm and 30 μm, 30 μm and 40 μm, 40 μm and 50 μm, 50 μm and 60 μm, 60 μm and 70 μm, 70 μm and 80 μm, 80 μm and 90 μm, 90 μm and 100 μm, or any other value therein or combination of ranges thereof, as suitable. In some examples, the minimal cross-sectional dimension 108 is shorter than the maximal cross-sectional dimension 110 such that the minimal cross-sectional dimension 108 may be less than 80%, 50%, 10%, 5%, 2%, 1%, 0.5%, 0.2%, or any other range or value therebetween, as suitable, as compared to the maximal cross-sectional dimension 110. In some examples, the maximal cross-sectional dimension 110 may be at least 20%, 50%, 100%, 200%, 300%, 400%, 500%, or any other range or value therebetween, as suitable, longer than the minimal cross-sectional dimension 108.

In some examples, the shape may have an indentation 113, or a concave portion, on one side or two opposing sides of the periphery 112 such that the thread 102 is at least partially crushed or compressed to define the noncircular cross-section. In some examples, the indentation 113 may be defined by a portion with a first cross-sectional dimension adjacent to one or more portions with a second cross-sectional dimension that is greater than the first cross-sectional dimension, in which case the first cross-sectional dimension may be considered a minimal cross-sectional dimension.

Also shown is an effective cross-sectional diameter 111 of the thread 102. This diameter 111 is determined by calculating a total area of the cross-section (as defined by the area inside the periphery 112) after which a diameter is calculated based on the total area based on the assumption that the total area pertains to that of a circle (shown in the figure as a corresponding circle 109 whose area is equal to the total cross-sectional area of the thread 102 and whose diameter is equal to the effective cross-sectional diameter 111). Therefore, the effective cross-sectional diameter 111 can be mathematically calculated using the following equation:

$$d = 2 \sqrt{A / π} \qquad ( Equation 1 )$$

where d is the effective cross-sectional diameter and A is the total cross-sectional area.

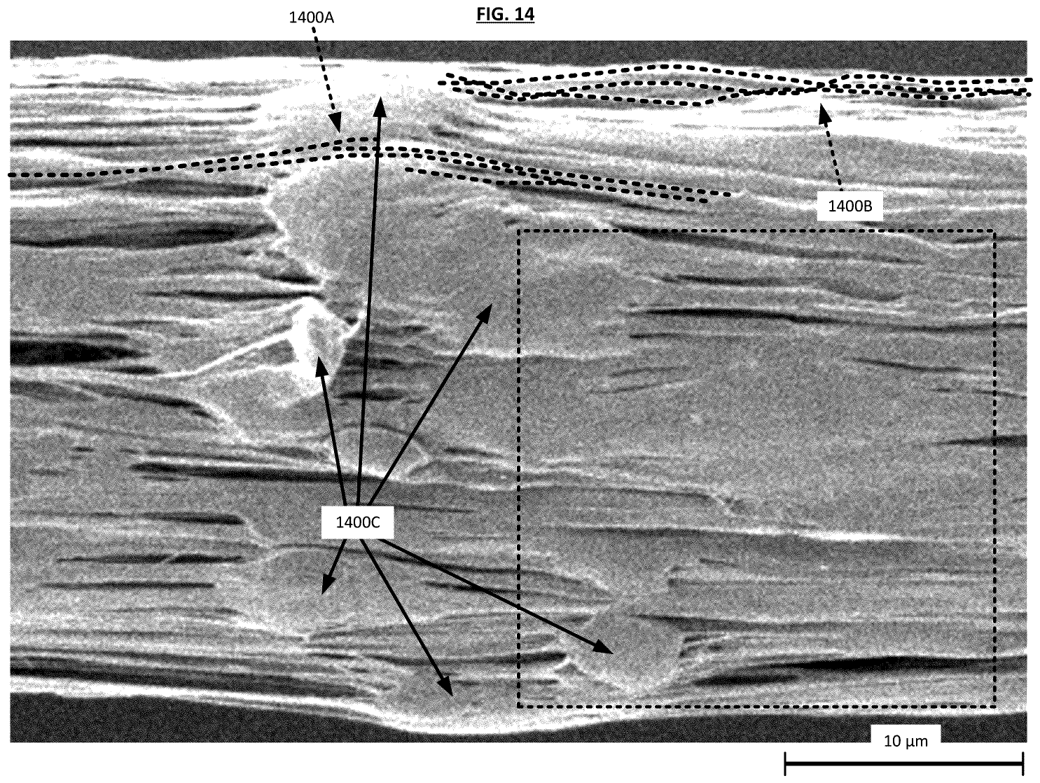

FIG. 1C illustrates another view of the cross-section of the thread 102, also showing a plurality of pore openings 114 (which may be formed as closed crevices located inside the thread 102, for example) in which pigment particles 116 can be disposed and contained, as well as two different surfaces such as a first surface 117 and a second surface 119 as defined by the periphery 112 with different curvatures or surface topology (such as surface shape and roughness). In some examples, the curvatures of the two surfaces may vary by a percentage such as at least 20%, 50%, 100%, 200%, 300%, 400%, 500%, or any other suitable value or range therebetween. In some examples, the periphery 112 or one or both of the surfaces 117, 119 may be defined by an irregular surface texture. The irregular surface texture may be uneven or coarse in texture. The irregular surface texture may include a plurality of fibers/fibrils of material separated by spaces. Generally, this irregular surface texture is due to the porous nature of the outer surface. In various embodiments, the surface of the thread 102 has generally the same porosity as the interior cross-section of the thread 102, or a close approximation thereto (see, e.g., FIG. 14 and the porous nature of the surface as shown). This porosity at the surface can be contrasted to other designs with non-porous outer surfaces or less porous outer surfaces. The pore openings 114 of the thread 102 may have any suitable shape or dimension such that the pore openings 114 are contained within the periphery 112 of the thread 102 such that the pigment particles 116 are contained within the periphery 112 of the thread 102 as well. In some examples, there may be additional pore openings extending inwardly from the periphery 112 (or outer surface) of the thread 102, but these additional pore openings are smaller than the pigment particles 116 so as to prevent the pigment particles 116 from leaking or escaping from inside the thread 102 and into the environment.

FIG. 1E shows the different surfaces of the thread 102 according to embodiments disclosed herein. For example, the thread 102 may include the first surface 117 (which may be referred to as an inner face) and the second surface 119 (which may be referred to as an outer face) with curvatures that are different from each other, such that the first surface 117 defines a first curvature C1-C1 that has a first center of curvature, and the second surface 119 defines a second curvature C2-C2 that has a second center of curvature different from the first center of curvature, as shown. In some examples, the thread 102 also has curved surfaces 124 connecting the first and second surfaces 117, 119 such as the first curved surface 124A and the second curved surface 124B on opposing sides of the thread 102, where the first curved surface 124A has a center of curvature that is different from a center of curvature for the second curved surface 124B, as shown. In some examples, the first and second centers of curvature for the first and second curved surfaces 124A, 124B may be different from a centroid 126 of the cross-section. The curved surfaces 124 may have a smaller radius (or radii) of curvature than the first and second surfaces 117 and 119. The radii of curvature may vary or differ by at least 20%, 50%, 100%, 200%, 300%, 400%, 500%, or any other suitable range or value therebetween, with respect to each other.

The thread 102 may be configured to take on a particular orientation when it is bent. In general terms, the flatter, or wider, aspect of the thread will tend to orient orthogonal to the bend, or curve direction whereas the more pointed, or narrow part of the thread will tend to orient parallel to the curve direction. In different terms, in some examples, the thread 102 assumes an inner-facing or inward-facing bias (as shown by the arrow) when the thread 102 is in a bent configuration, where the bias extends along the axial length of the thread 102). The bias may be directed toward the center of curvature of either the first surface 117 or the second surface 119, for example. The bent portion of the surgical suture may form part of a knot.

The thread 102 of the suture 100 has a specific range of cross-sectional dimensions (size) as explained herein. Generally, sizes of surgical sutures are defined by the USP standards as previously described.

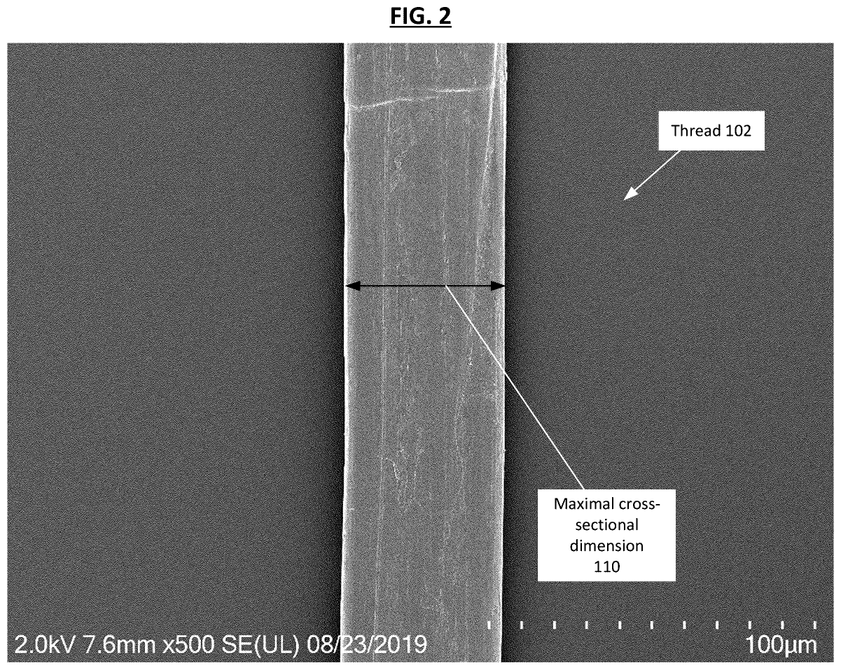

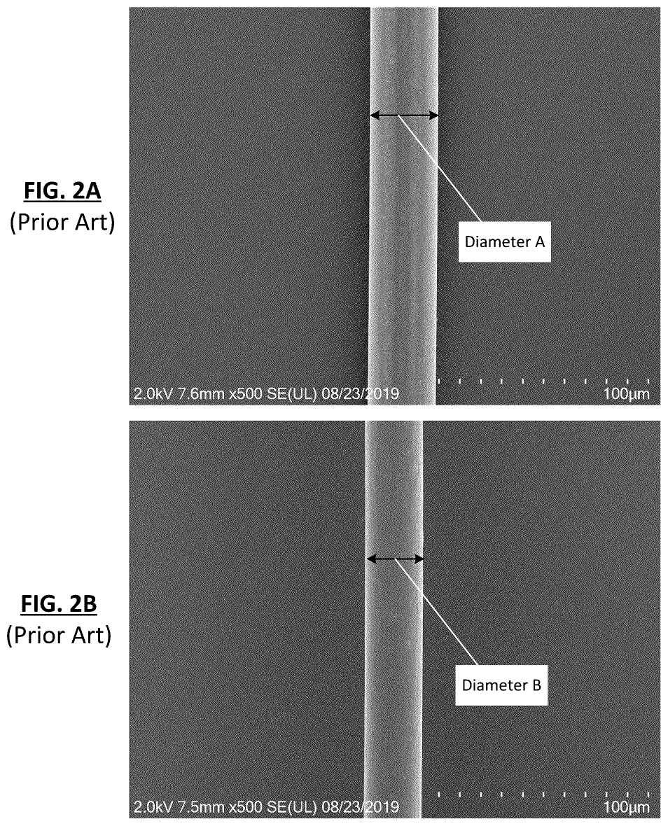

FIGS. 2, 3, 4, 5, 6, and 7 show scanning electron microscope (SEM) images of the thread 102 as viewed from two different angles, according to embodiments disclosed herein. In comparison, FIGS. 2A, 3A, 4A, 5A, 6A, and 7A show SEM images of a prior-art ETHILON® nylon suture (sold by Ethicon, Inc.; Raritan, NJ), and FIGS. 2B, 3B, 4B, 5B, 6B, and 7B show SEM images of a prior-art ETHILON® polypropylene (PP) suture (sold by Ethicon, Inc.; Raritan, NJ) viewed from similar angles and magnifications.

FIGS. 2, 2A, and 2B are images taken at 500× magnification, with a scale showing the length of 100 μm relative to the image (such that a distance between two consecutive vertical markers represents 10 μm). Indicated at the bottom of the images are: “2.0 kV 7.6 mm×500 SE (UL) Aug. 23, 2019” for FIGS. 2 and 2A, and “2.0 kV 7.5 mm×500 SE (UL) Aug. 23, 2019” for FIG. 2B.

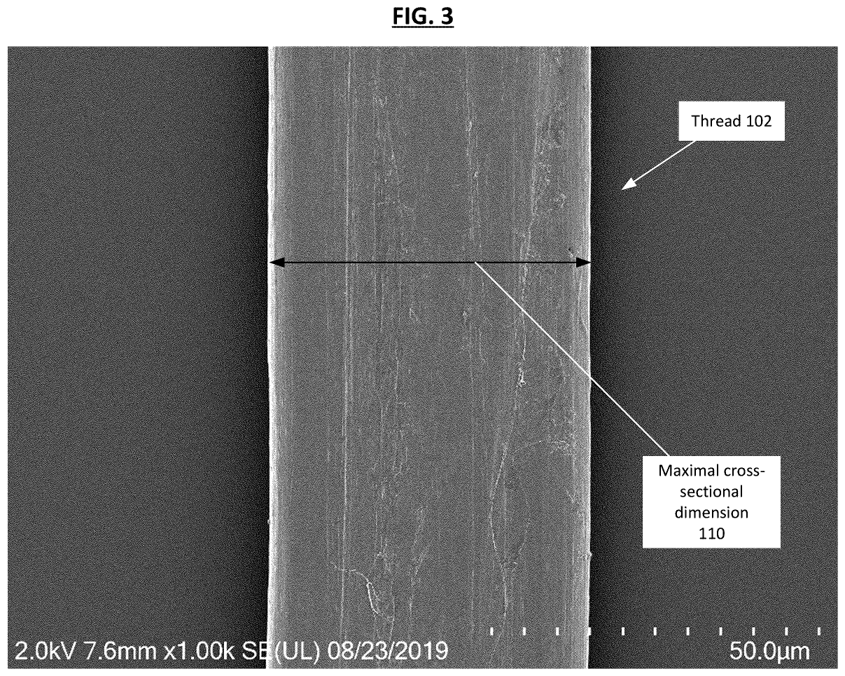

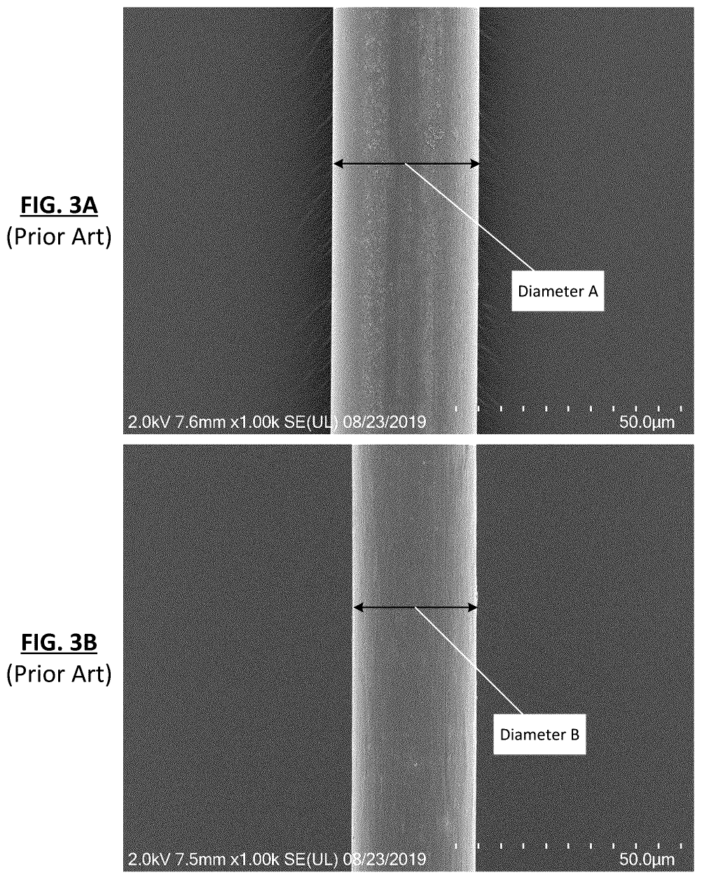

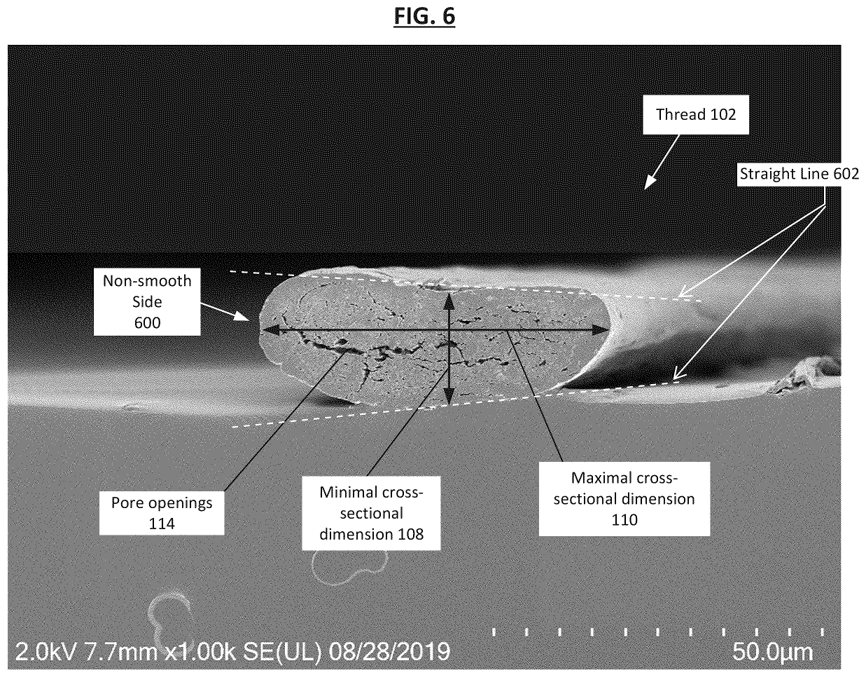

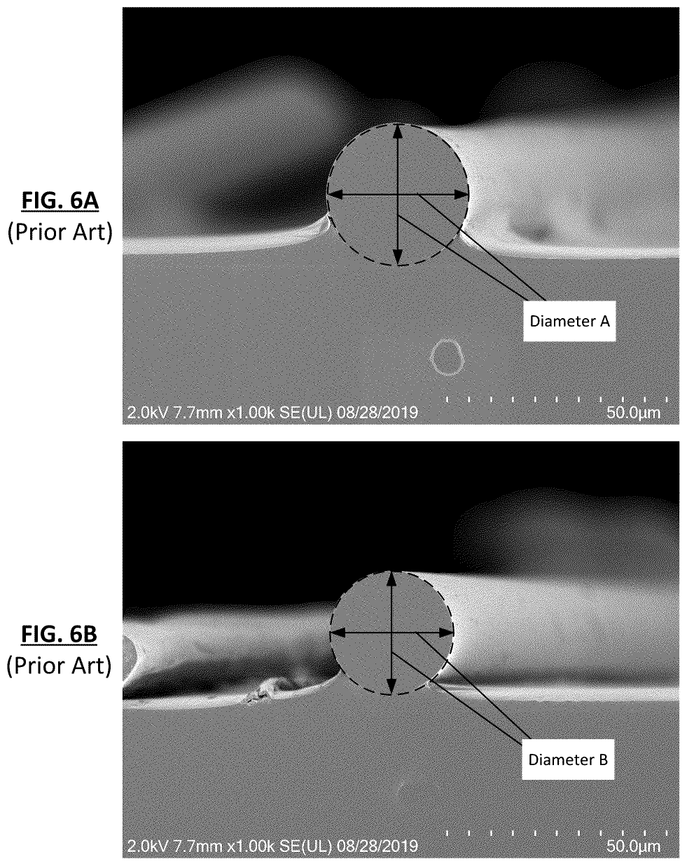

FIGS. 3, 3A, and 3B as well as FIGS. 6, 6A, and 6B are images taken at 1000× magnification, with a scale showing the length of 50.0 μm relative to the image (such that a distance between two consecutive vertical markers represents 5.0 μm). Indicated at the bottom of the images are: “2.0 kV 7.6 mm ×1.00k SE (UL) Aug. 23, 2019” for FIGS. 3 and 3A, “2.0 kV 7.5 mm ×1.00k SE (UL) Aug. 23, 2019” for FIG. 3B, and “2.0 kV 7.7 mm ×1.00k SE (UL) Aug. 28, 2019” for FIGS. 6, 6A, and 6B.

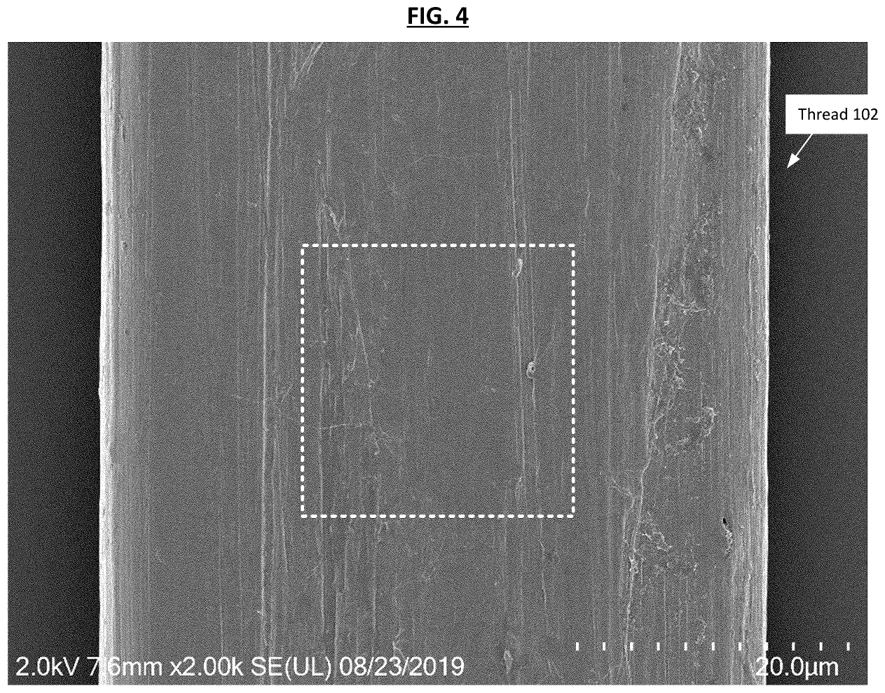

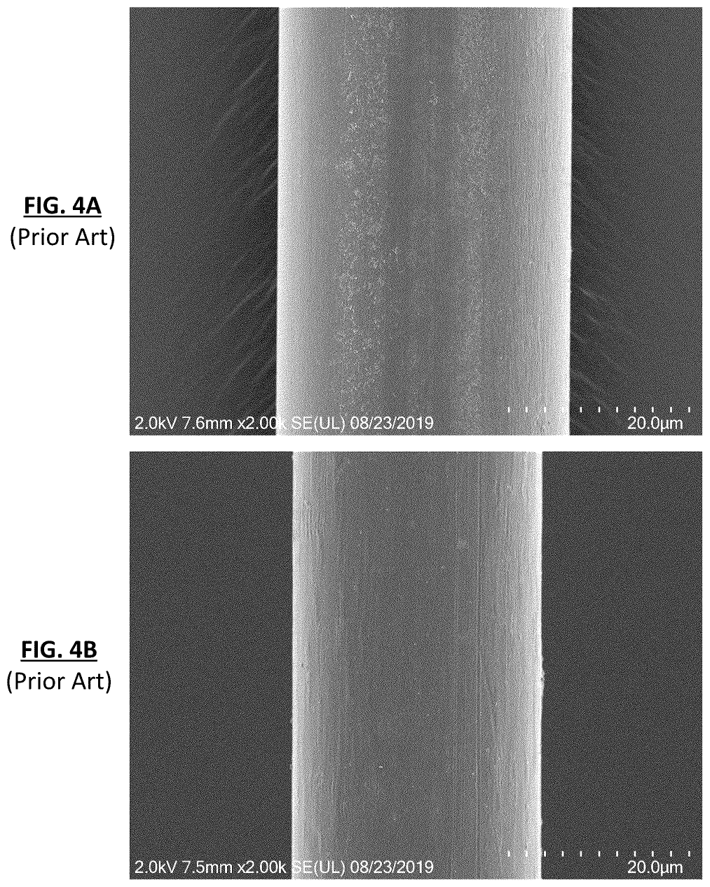

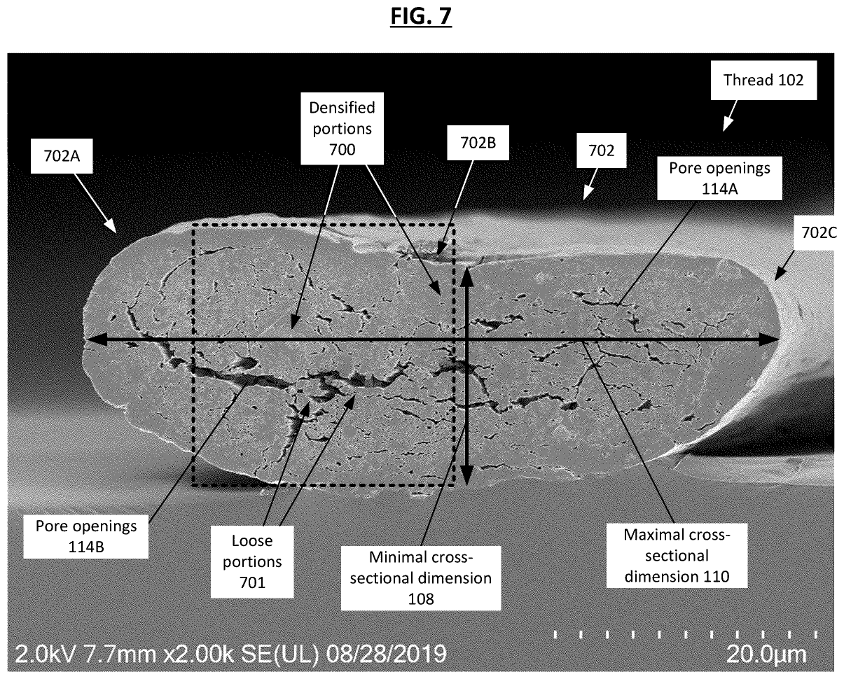

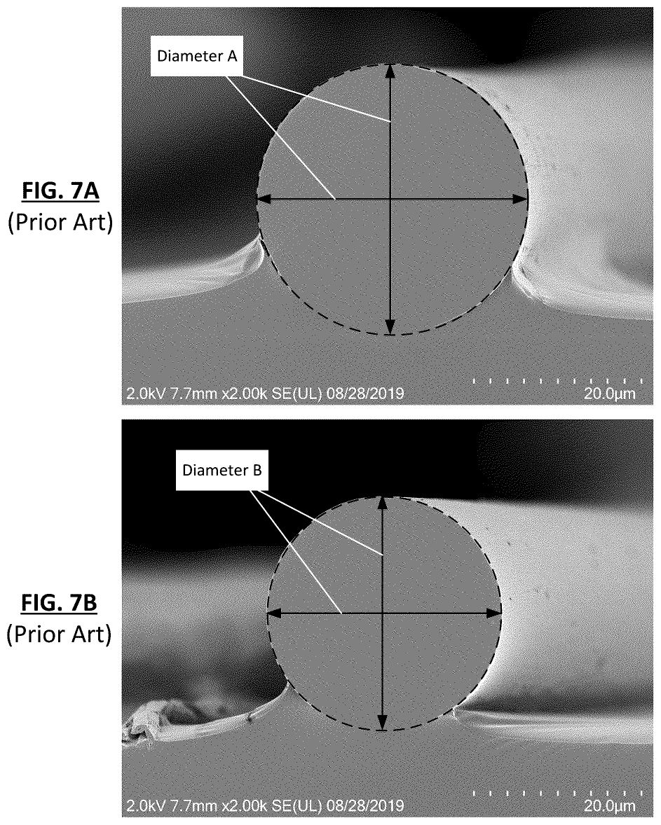

FIGS. 4, 4A, and 4B as well as FIGS. 7, 7A, and 7B are images taken at 2000× magnification, with a scale showing the length of 20.0 μm relative to the image (such that a distance between two consecutive vertical markers represents 2.0 μm). Indicated at the bottom of the images are: “2.0 kV 7.6 mm ×2.00k SE (UL) Aug. 23, 2019” for FIGS. 4 and 4A, “2.0 kV 7.5 mm ×2.00k SE (UL) Aug. 23, 2019” for FIG. 4B, and “2.0 kV 7.7 mm ×2.00k SE (UL) Aug. 28, 2019” for FIGS. 7, 7A, and 7B.

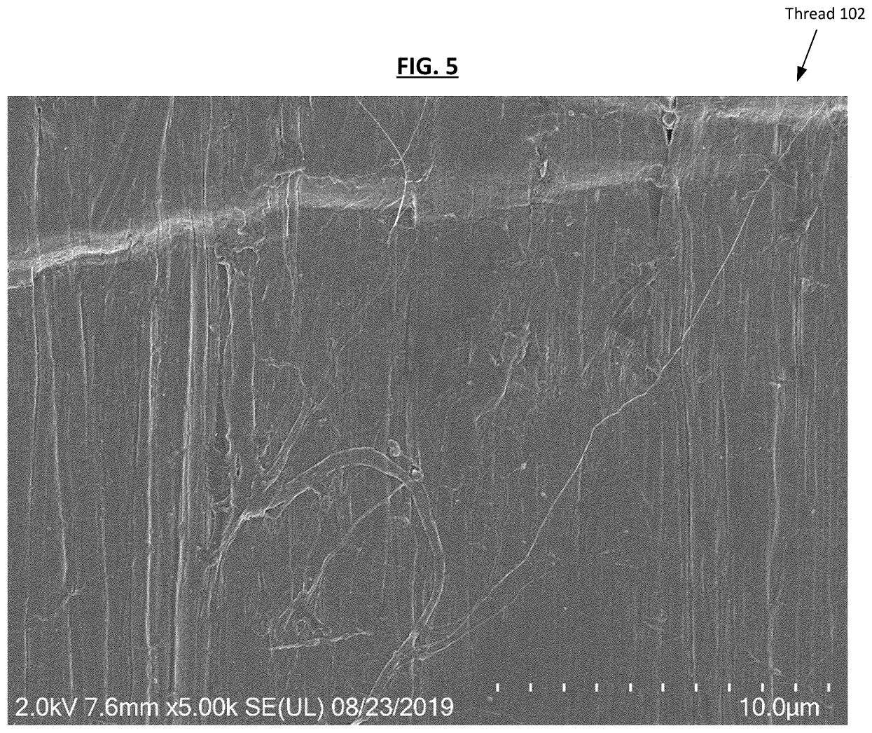

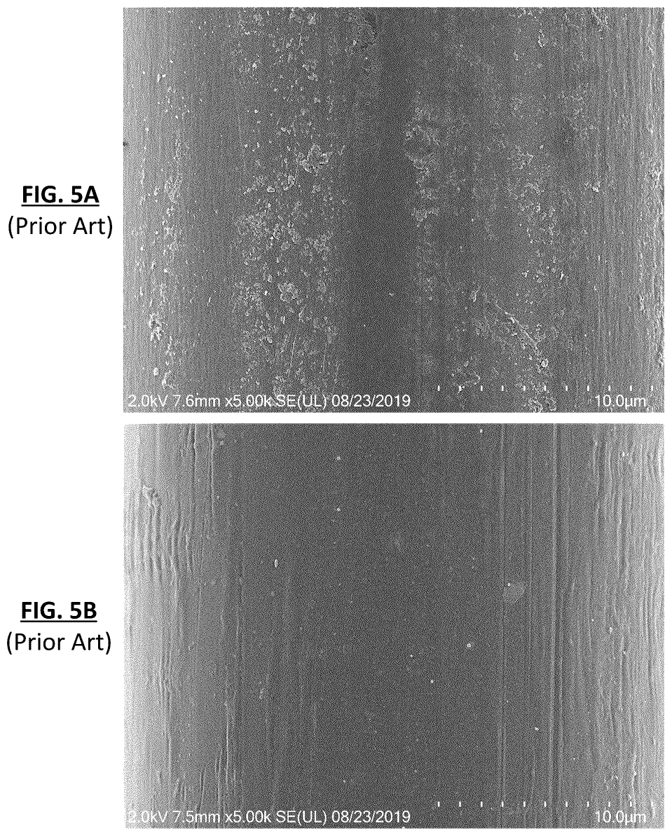

FIGS. 5, 5A, and 5B are images taken at 5000× magnification, with a scale showing the length of 10.0 μm relative to the image (such that a distance between two consecutive vertical markers represents 1.0 μm). Indicated at the bottom of the images are: “2.0 kV 7.6 mm ×5.00k SE (UL) Aug. 23, 2019” for FIGS. 5 and 5A, and “2.0 kV 7.5 mm ×5.00k SE (UL) Aug. 23, 2019” for FIG. 5B.

It can be observed based on these figures that the ETHILON® nylon suture and the ETHILON® PP suture both have a substantially (if not near-perfectly) circular cross-section as shown by the broken lines of FIGS. 6A, 6B, 7A, and 7B which represent the circular peripheries of these sutures, whereas the thread 102 has an oblong cross-sectional shape. As such, in the ETHILON® sutures, the minimal and maximal cross-sectional lengths would be substantially similar to each other and define the diameter for the appropriate suture. As such, the ETHILON® nylon suture has a cross-sectional diameter of “Diameter A” and the ETHILON® PP suture has a cross-sectional diameter of “Diameter B” as labeled. Based on the scales, the value of Diameter A can be approximated at about 33 μm, and the value of Diameter B can be approximated at about 28 μm. In comparison, as measured in FIGS. 6 and 7, the minimal cross-sectional dimension 108 of the thread 102 is about 18 μm, and the maximal cross-sectional dimension 110 of the thread 102 is about 53 μm.

In some examples, the thread 102 as shown in FIG. 7 may be a synthetic fiber having an axial length suitable for tissue ligation or approximation and a cross-section disposed orthogonally to the length at a portion of the axial length. The cross-section may include one or more densified areas 700 surrounding openings disposed between the densified areas 700, such as the pore openings 114. For example the pore openings 114A and 114B may be present, where the pore openings 114A are smaller than the pore openings 114B, or the pore openings 114B have larger average pore sizes than the pore openings 114A. The openings may change size depending upon whether the thread 102 is under an external force (e.g., compression, tension, and/or torsional forces). The openings may be collapsible when the synthetic fiber is externally compressed in a plane of the cross-section, for example. The openings (e.g., pore openings) may be present in a first uncompressed configuration in the absence of external forces being applied to the fiber (or thread 102), and the openings may be present in a second compressed configuration in response to the external force being applied to the fiber, either at the same size or at a reduced size as previously referenced. The fiber may have a minimal cross-sectional dimension and a maximal cross-sectional dimension such that at least one of the minimal cross-sectional dimension or the maximal cross-sectional dimension is reduced by at least 30% in response to the fiber transitioning from the first uncompressed configuration to the second compressed configuration.

The pore openings 114 define a plurality of loose or distributed portions 701 which are less densely configured as compared to the densified portions 700. The densified (first) portions 700 may define a first porosity, and the distributed (second) portions 701 may define a second porosity that is greater than the first porosity. In some examples, an outer region closer to the periphery of the thread 102 may have lower porosity and greater density than in an inner region farther from the periphery of the thread 102. Beneficially, the variation in density and porosity within the thread 102 facilitates the load to be redistributed when the thread is tied into a knot, thereby spreading the load at the knot to reduce the stress applied to the knot section. The variation in density and porosity also improves the foldability of the thread so as to reduce, minimize, or eliminate any permanent or irreversible damage that may be experienced by the thread at the folded area. For example, the thread is better able to accommodate the natural compression that occurs during bending or folding due to the selective ability of different portions of the cross-section of the thread to be compactible.

In some examples, the synthetic fiber may have side surfaces 702 each extending axially along at least a portion of the axial length of the synthetic fiber. The axially-extending side surfaces may include a substantially flat surface 702B disposed between two curved surfaces 702A and 702C. A flat surface may be defined by a straight line disposed over the surface that generally tracks the surface over a distance, with equal portions of the surface being disposed on each side of the straight line until reaching a location along the distance where the surface terminates or significantly deviates away from the straight line. In other terms, a substantially flat surface (or flat side) of a synthetic surgical fiber may be defined as a surface that is flat or generally flat, and that may include slightly concave or convex portions within the flat surface as illustrated in various examples (see FIG. 6 at straight line 602). In some examples, a substantially flat surface can be defined by a relatively large radius of curvature exceeding the radius of the suture and presenting a nearly flat curvature. Such examples can be illustrated with a radius of curvature that is greater than 1 mm, greater than 2 mm, greater than 3 mm, or any other suitable value or range therebetween, or with any suitable range or value that is greater than any maximum cross-sectional dimension of the suture. In some similar examples, a substantially flat surface of the synthetic surgical fiber may be defined as a surface with a radius of curvature that is at least 3 times, at least 4 times, at least 5 times, at least 10 times, at least 20 times, at least 30 times, at least 40 times, at least 50 times, at least 100 times any maximum cross-sectional dimension of the suture, or with any other suitable value or range therebetween, with respect to a maximal cross-sectional length (or an edge-to-edge distance as measured transversely with respect to a longitudinal axis) of the fiber. Each of the two curved surfaces 702A and 702C may curve about the axis from 90 degrees to 180 degrees, for example. In some examples, the cross-section defines the flat surface 702B of the synthetic fiber extending along the portion of the axial length, such that the synthetic fiber may be predisposed to twist about the axis to present the flat surface 702B as an inner surface of a curvature formed when the fiber is deformed to form the curvature.

In some examples, the thread 102 as shown in FIG. 6 or FIG. 7 may be a synthetic fiber having an axial length suitable for tissue ligation or approximation. The fiber may be a suture that meets the USP class I size 4-0 or smaller nonabsorbable surgical suture requirements. The thread 102 may have a transverse cross-section including densified areas (for example the densified portion 400) surrounding the openings 114 that are disposed between the densified areas. The openings 114 may be collapsible when the thread 102 is transversely compressed. The thread 102 may be compliant and bendable to a bending radius of approximately 0.19 mm when two ends of the suture are held under tension by a pair of weights totaling approximately 100 mg in mass.

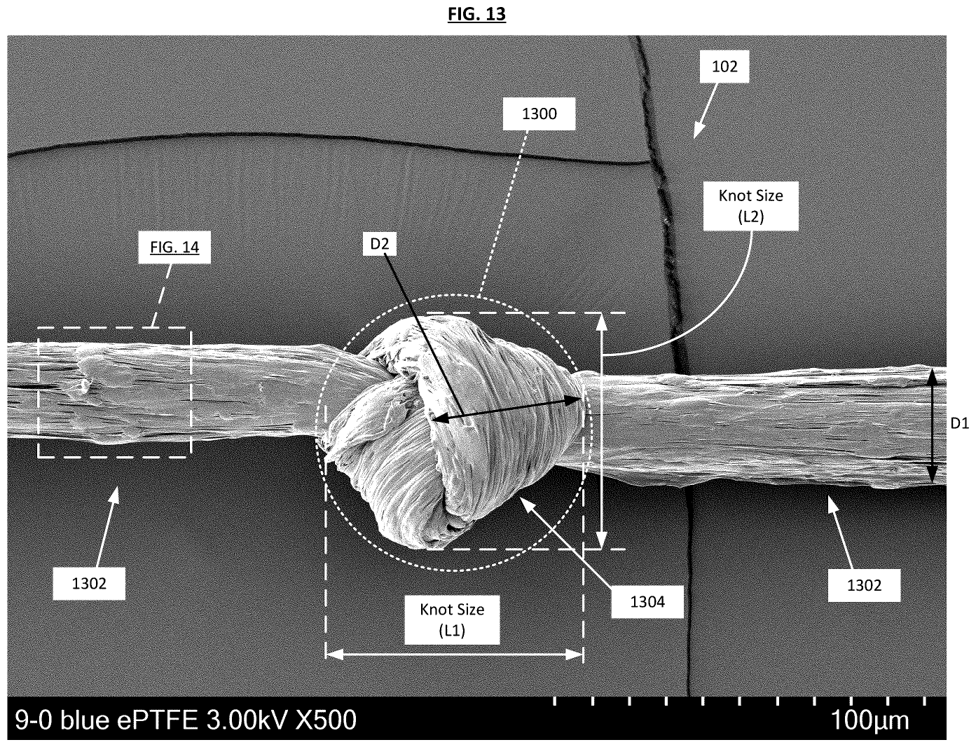

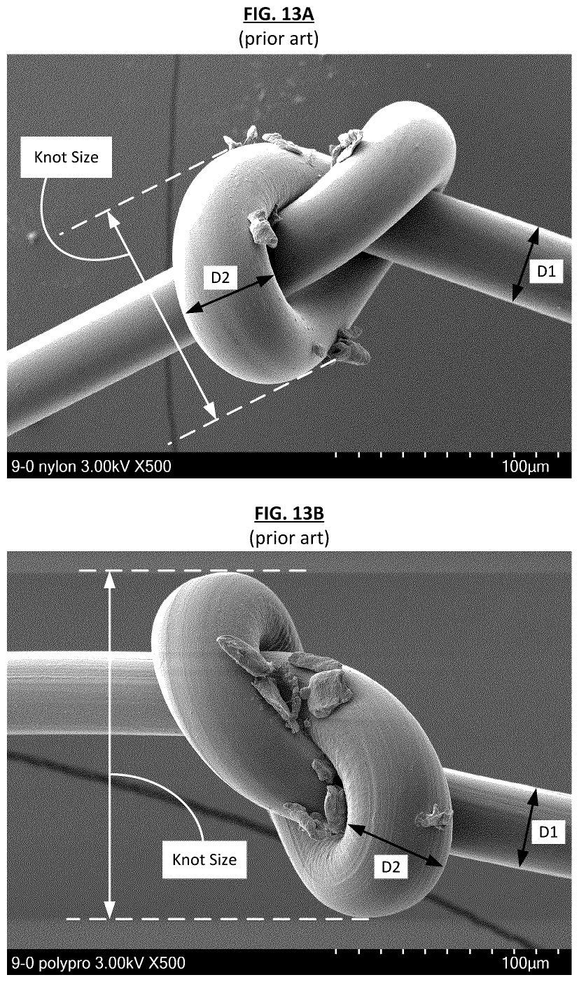

In some examples, the thread 102 may have a variable cross-section that is defined along a portion of the axial length of the thread 102. A first, unknotted portion of the axial length of the thread 102 may have an uncompressed state (for example, uncompressed state 1302 as shown in FIG. 13) in which the first, unknotted portion presents a first cross-section having a first peripheral edge (D1) circumscribing the thread 102. In some examples, the peripheral edge may be measured when the thread 102 is under sufficient external force to be in a taut or straight configuration, or when the thread is in a curved or relaxed configuration that is not taut or straight (such as in the absence of such external force). A second, knotted portion of the axial length of the thread 102 may have a compressed state (for example, compressed state 1304 as shown in FIG. 13) in which the second, knotted portion presents a second cross-section having a second peripheral edge (D2) circumscribing the thread 102. The second peripheral edge D2 may have a different (for example, greater) length as compared to the first peripheral edge D1, and the first, unknotted portion of the axial length of the thread 102 is located adjacent the second, knotted portion. In some examples, the variable cross-section of the thread 102 may vary between an uncompressed state 1302 presenting a first cross-section having first openings (such as the openings 114B, for example) disposed within the thread 102 at the first cross-section, and a compressed state 1304 presenting a second cross-section having second openings (such as the openings 114A, for example) disposed within the thread 102 at the second cross-section. The second openings may have a reduced size compared to the first openings, and the compressed state may be present in the thread 102 within a knot 1300 that is formed by the thread 102 in a knotted configuration, such that the uncompressed state is present in the thread 102 adjacent to the knot 1300. In some examples, in the knotted configuration, the thread 102 has a first portion defining a first porosity and a second portion defining a second porosity greater than the first porosity, where the first portion corresponds to the knotted portion and the second portion corresponds to an unknotted portion. The first porosity may be defined by first pore openings, and the second porosity may be defined by second pore openings having larger average pore sizes than the first pore openings.

In some examples, referring back to FIG. 1C, the suture 100 may be a sterilized nonabsorbable surgical suture which includes a polymeric fiber (that is, the thread 102 may comprise the polymeric fiber) having a non-circular cross-section with a maximal cross-sectional dimension and an orthogonally disposed minimal cross-sectional dimension that is less than the maximal cross-sectional dimension, such that the minimal cross-sectional dimension is from 0.010 mm to 0.049 mm. The suture 100 may also include a coating 118 disposed on the fiber, for example coated on the periphery 112 of the thread 102, such that the coating 118 is disposed to not significantly add to the maximal cross-sectional dimension or the minimal cross-sectional dimension of the fiber or thread 102. In some examples, the coating 118 may be hydrophilic. In some examples, the thread 102 may be made of a monofilament polymeric fiber, i.e., the fiber may be monofilament.

In some examples, when the minimal cross-sectional dimension is in a range from 0.010 mm to less than 0.020 mm, the surgical suture 100 has an average straight-pull tensile strength that is equal to or greater than 0.006 kgf. In some examples, when the minimal cross-sectional dimension is in a range from 0.020 mm to less than 0.030 mm, the surgical suture 100 has an average straight-pull tensile strength that is equal to or greater than 0.019 kgf. In some examples, when the minimal cross-sectional dimension is in a range from 0.030 mm to less than 0.040 mm, the surgical suture 100 has an average straight-pull tensile strength that is equal to or greater than 0.043 kgf. In some examples, when the minimal cross-sectional dimension is in a range from 0.040 mm to less than 0.050 mm, the surgical suture 100 has an average knot-pull tensile strength that is equal to or greater than 0.060 kgf.

In some examples, the synthetic fiber may have an average tensile strength when the fiber is subjected to a tensile strength measurement technique set forth in USP Section 881 entitled “Tensile Strength”. In some examples, the synthetic fiber may have a tensile strength that is determined by a tensile strength measurement technique involving the disposition of the fiber between two clamps with at least one of the two clamps moving away from the other to apply a measurable tensile force to the suture. In some examples, the synthetic fiber may be disposed to form a simple knot (see for example a tensioned knot 1300 shown in FIG. 13) along a portion of the axial length prior to being subjected to a tensile strength measurement technique. The simple knot may form a loop through which a portion of the fiber passes through the loop to form the simple knot when respective ends of the fiber are pulled away from each other. In some examples, the synthetic fiber may have a tensile strength that is determined from a tensile strength measurement technique, and a USP tensile strength of the surgical suture may further be determined by averaging the fiber tensile strength with a plurality of additional similar measurements obtained from at least nine additional samples of the surgical suture. In some examples, the synthetic fiber may have a fiber tensile strength that is determined with a tensile strength measurement technique, and the surgical suture may have a USP tensile strength value determined from the fiber tensile strength averaged with additional similar fiber tensile strengths obtained from at least nine additional samples of the surgical suture. Accordingly, the USP tensile strength value may be a straight-pull tensile strength of at least 0.01 N for USP size 12-0, at least 0.06 N for USP size 11-0, at least 0.186 N for USP size 10-0, at least 0.422 N for USP size 9-0, and at least 0.59 N for USP size 8-0.

The maximal cross-sectional dimension may be at least 20% greater than the minimal cross-sectional dimension. The surgical suture 100 may include ePTFE as the polymeric fiber, for example, and in some examples, the polymeric fiber may be microporous. The microporosity may vary among different examples, such as from 35% to 55% in porosity, or any one or more of from 35% to 40%, from 40% to 45%, from 45% to 50%, and/or from 50% to 55%, as well as any suitable value or range therebetween, or any suitable combination of ranges thereof. In some examples, the surgical suture 100 may be colored using pigment particles 116 as shown. In some examples, the surgical suture has an MTS of at least 70 kgf/mm², at least 75 kgf/mm², at least 80 kgf/mm², at least 85 kgf/mm², at least 90 kgf/mm², at least 95 kgf/mm², at least 100 kgf/mm², at least 105 kgf/mm², at least 110 kgf/mm², or any suitable value or range therebetween.

In some examples, the surgical suture 100 is compliant and bendable to a bending radius of between about 0.19 mm and about 0.50 mm when two ends of the suture are held under tension by a pair of weights totaling about 100 mg in mass. Such methods of testing the compliance and bendability of the suture by measuring its bending radius are further explained in detail below. In some examples, the compliant surgical suture 100 has a knot size of between about 50 μm and about 100 μm, the measurement method of which is also explained in detail below. In some examples, the surgical suture 100 has a coefficient of friction equal to or less than approximately 0.02, the measurement method of which is also explained in detail below.

In some examples, the suture 100 may be comparable to USP size 12-0, 11-0, 10-0, 9-0, or 8-0 as further explained herein. In some examples, the suture 100 conforms with the requirements of the United States Pharmacopeia published by U.S. Department of Health and Human Services, Food and Drug Administration, Center for Devices and Radiological Health as “Surgical Sutures-Performance Criteria for Safety and Performance Based Pathway: Guidance for Industry and Food and Drug Administration Staff” and published on Apr. 11, 2022, as referenced herein.

FIG. 1D shows an example of the suture 100 as explained above which is included in a surgical suture kit or package 120, where the kit or package 120 includes a label 122 which accompanies the suture 100 (for example, pasted or printed on the surface of the kit or package 120) explaining or stating that the accompanying surgical suture is at least one of USP size 12-0, 11-0, 10-0, 9-0, or 8-0, as appropriate. A pair of forceps is also shown for the doctor or physician to use in order to take the suture 100 out from inside the kit or package 120.

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

### Manufacturing Process

The suture may be formed or manufactured using any of a variety of means, including those disclosed herein. For example, the material (as disclosed herein) to be used for the thread portion of the suture may be provided utilizing an extrusion process with any of a variety of pieces of suitable suture extrusion equipment to form the filaments to be used as the fiber. Generally, manufacturing techniques will be compliant with applicable regulations, such as the those of the Food and Drug Administration (FDA), and in accordance to the manufacturing and testing guidelines for the applicable industry, such as those provided by USP.

### Test Methods

It should be understood that although certain methods and equipment are described below, other methods or equipment determined suitable by one of ordinary skill in the art may be alternatively utilized.

Matrix tensile strength (MTS) of a suture may be determined using the method as follows. Tensile break load is measured using an INSTRON 122 tensile test machine equipped with flat-faced grips and a 0.445 kN load cell. The gauge length is about 5.08 cm and the cross-head speed is about 50.8 cm/min. The sample dimensions are about 2.54 cm by about 15.24 cm. For highest strength measurements, the longer dimension of the sample is oriented in the highest strength direction. For the orthogonal MTS measurements, the larger dimension of the sample is oriented perpendicular to the highest strength direction. Each sample is weighed using a Mettler Toledo Scale Model AG204, then the thickness is measured using the Kafer FZ1000/30 snap gauge; alternatively, any suitable means for measuring thickness may be used. The samples are then tested individually on the tensile tester. Three different sections of each sample are measured. The average of the three maximum loads (i.e., peak force) measurements is calculated. The longitudinal and transverse matrix tensile strengths (MTS) are calculated using the following equation: MTS=(maximum load/cross-section area)*(bulk density of PTFE)/(density of the porous membrane), where the bulk density of the PTFE is taken to be about 2.2 g/cm³.

Porosity of a specimen such as the suture material may be accounted for by multiplying the tensile strength by the ratio of density of the polymer to the density of the specimen. Porosity may be assessed using an air permeability test method of a sample according to the general teachings of ISO 5636-5 by measuring the ability of air to flow through a material as follows. A test specimen of leaflet or leaflet material is placed in a Gurley densometer (Model 4110 Gurley Precision Instruments, Troy, N.Y.) set up with the 0.25 inch² (1.61 cm²) orifice. The time to flow 100 cc of air through the sample is measured and divided by 4 to obtain the Gurley time in seconds. A Gurley time above about 1000 seconds indicates that the sample is impermeable to air and is judged to be impermeable according to the definitions in this specification.

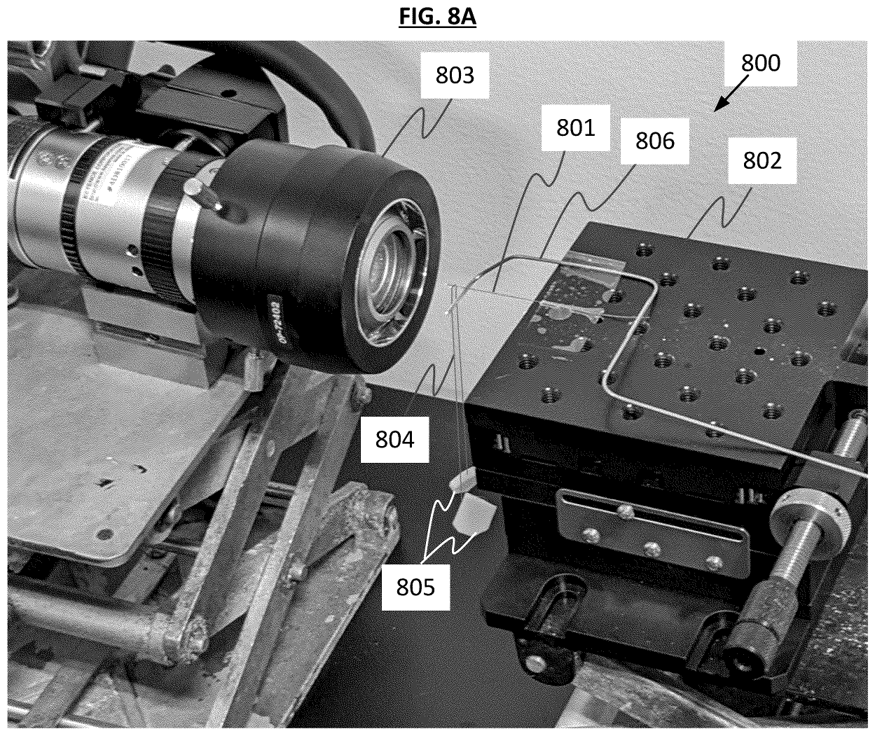

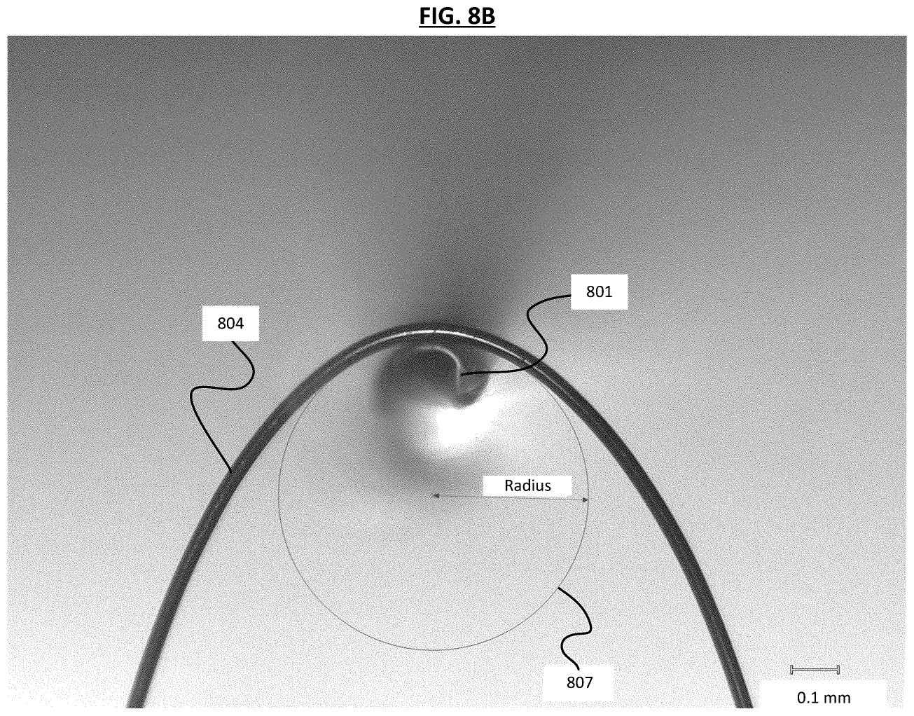

The “curvature radius”, or the “bending radius”, of a suture may be measured according to a measurement setup or test apparatus 800 shown in FIG. 8A. The test apparatus 800 is developed to apply a testing method to characterize the bending stiffness of a suture. In the testing method, light weights are attached to either end of a suture and the suture is draped over a round mandrel. Using a microscope, an image of the suture is captured where it hangs from the mandrel and the bend radius that the suture takes is measured and recorded. A suture with a large bend radius is stiffer than a suture with a small bend radius when tested under the same conditions.

The test apparatus 800 comprises a round mandrel 801 with a diameter of 250 μm attached to a stage 802 that is adjustable in the x, y, and z directions, as shown in FIG. 8A. The mandrel 801 is oriented so that the longitudinal axis is horizontal with respect to the ground such that the axis extends from the edge of the stage 802. A microscope 803 (which in the image is a Keyence VHX6000 microscope) is oriented horizontally so that the optical axis of the microscope 803 aligns with the long axis of the mandrel 801.

The suture 804 to be tested is prepared with a sufficient length with two weights 805 attached to both ends of the suture 804. The two weights may be of approximately equal mass or weight. For example, the weights 805 may be made of vinyl foam or any other suitable material. The suture 804 and weights 805 are placed on a balance and the mass is measured. For example, the mass of the suture 804 may be about 0.0001 g (or 100 micrograms). The weight 805 may be adjusted by trimming or adding a portion until the target weight is achieved. The measured weight may be within about 0.001 g (or 1 mg) of the target mass. The target mass of the weights 805 may be about 50 mg, about 100 mg, or any suitable value or range therebetween.

The suture 804 with the desired weights 805 is carefully draped over the mandrel 801 as shown, ensuring that the weights 805 hang freely below without contacting any portion of the apparatus 800. Alignment bar 806 is positioned behind the suture 804 so that it gently contacts both dangling ends of the suture 804, ensuring that the plane of the arc created by the suture 804 is substantially perpendicular to the optical axis of the microscope 803. The x, y, and z stages (e.g., components of the stage 802 that is adjustable in these directions) are used to center and focus the image.

FIG. 8B shows an exemplary image of a suture 804 under test as captured by the microscope 803. The circle 807 represents the bending radius (shown as “radius”) assumed by the suture 804 as it rest on mandrel 801. Note that the end of mandrel 801 appears out of focus in this image because it is not in the same focal plane as the suture 804. The radius of circle 807 is reported for each suture 804 tested under each condition. At the bottom right hand corner of the image is a scale for 0.1 mm length with respect to the image.

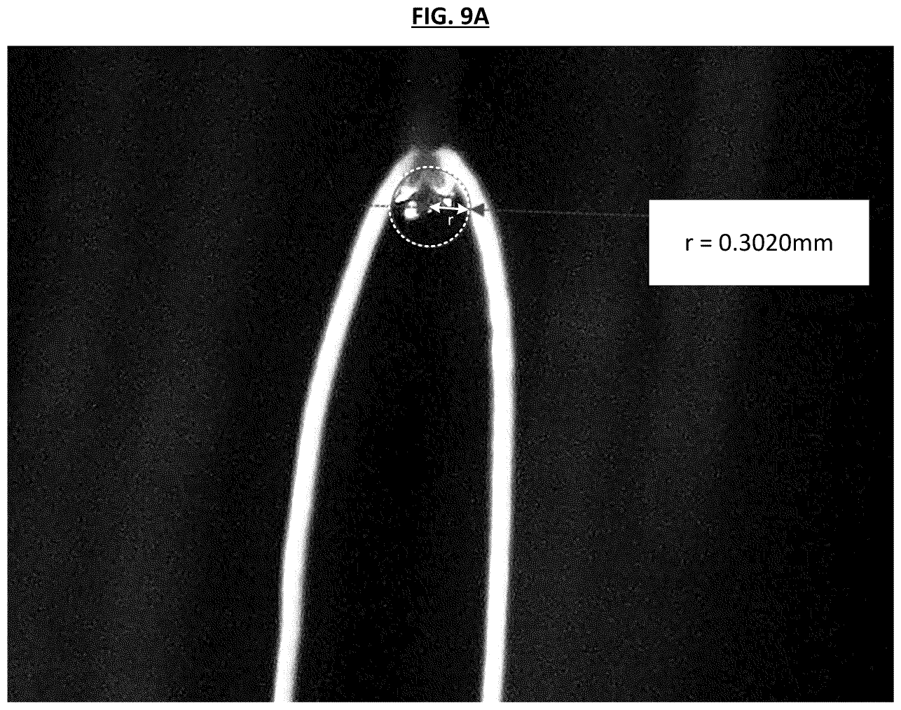

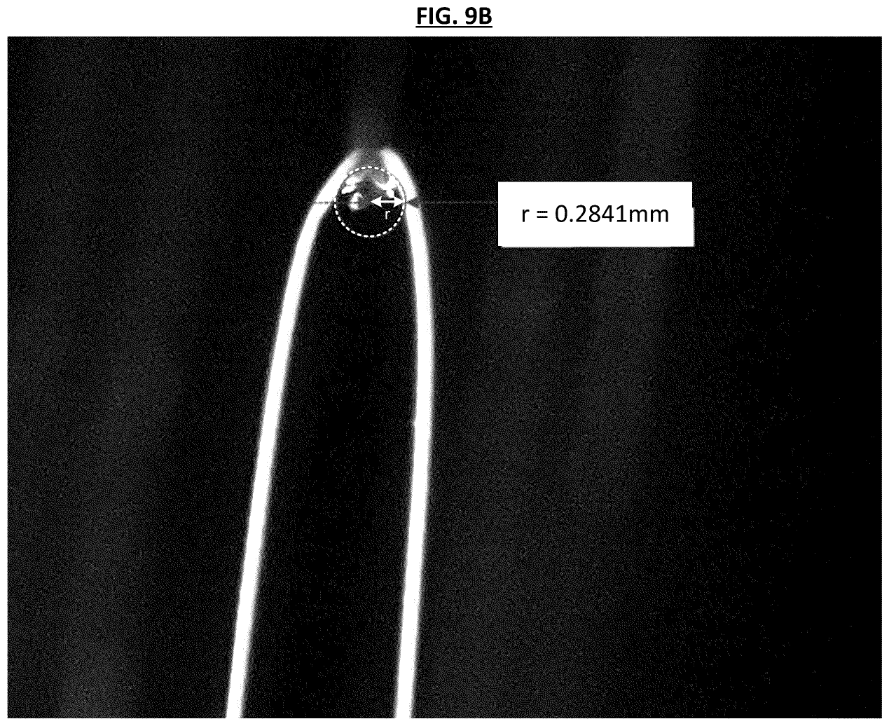

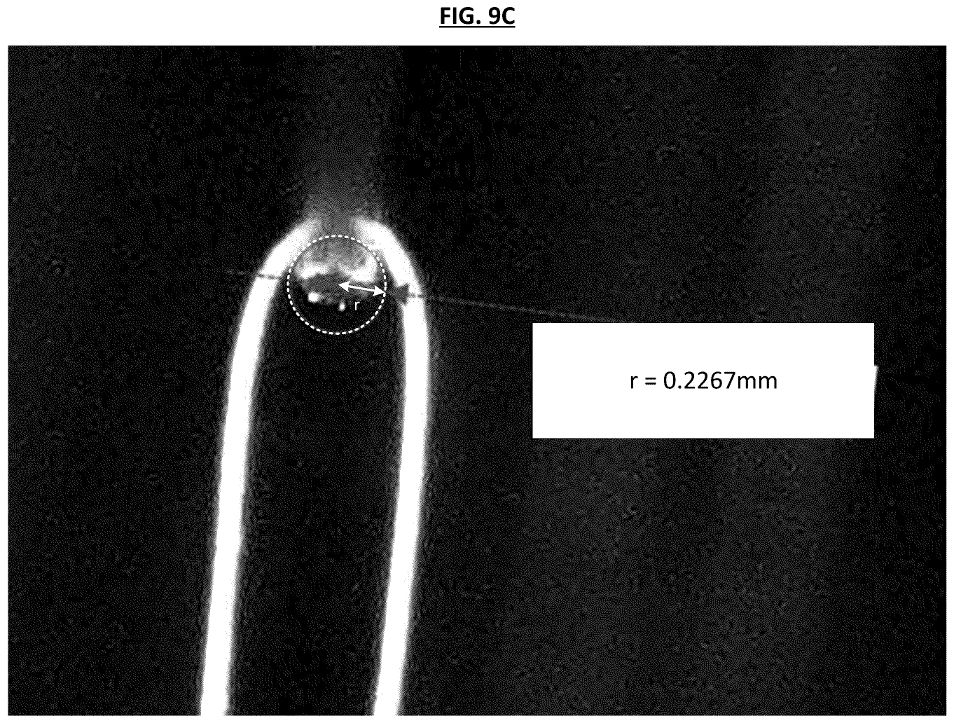

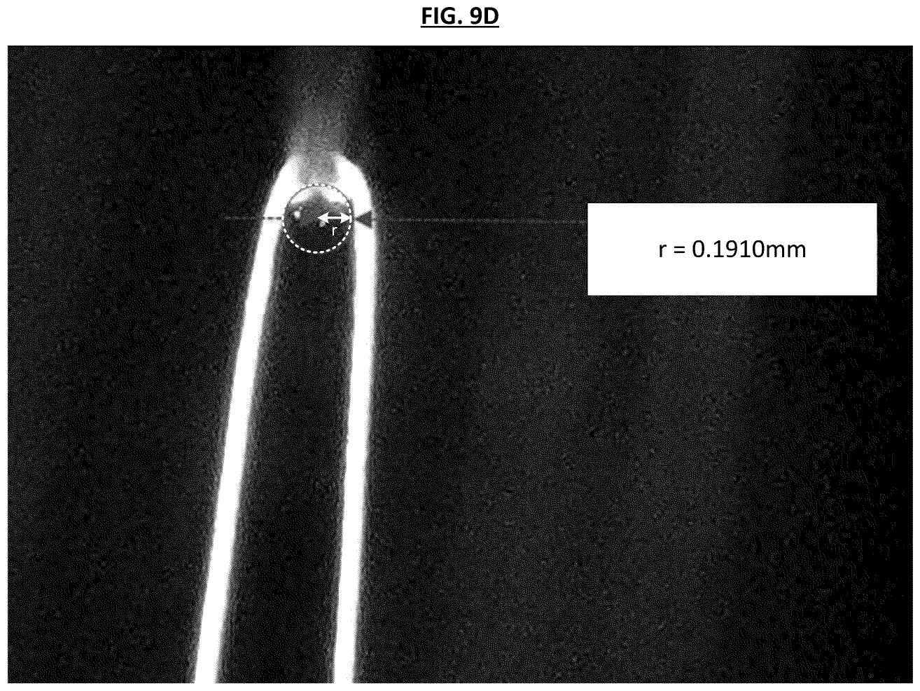

FIGS. 9A through 9D are exemplary images of the suture 100 that are captured by the microscope 803 under different weights 805. FIGS. 9A and 9B are two samples of the suture 100 whose images are taken when 47 mg of weights 805 are used in the test method shown in FIG. 8A. FIGS. 9C and 9D are two samples of the suture 100 whose images are taken when 94 mg of weights 805 are used. Therefore, the measured radii (“r”) of FIGS. 9A and 9B (0.3020 mm and 0.2841 mm, respectively) are greater than the measured radii of FIGS. 9C and 9D (0.2267 mm and 0.1910 mm, respectively.)

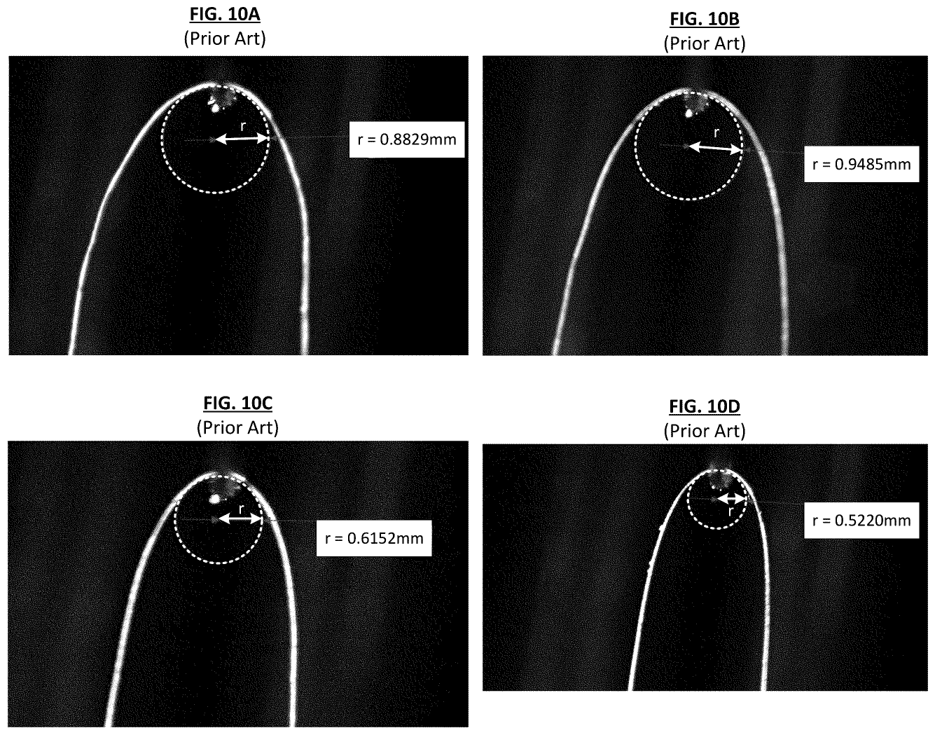

FIGS. 10A through 10D are exemplary images of a USP 9-0 nylon suture that are captured by the microscope 803 under similar testing conditions as FIGS. 9A through 9D, provided for comparison. For example, in FIGS. 10A and 10B, the USP 9-0 nylon suture has the measured radii (“r”) of 0.8829 mm and 0.9485 mm, respectively, when 47 mg of weights 805 are used (compare to FIGS. 9A and 9B), and in FIGS. 10C and 10D, the USP 9-0 nylon suture has the measured radii (“r”) of 0.6152 mm and 0.5220 mm, respectively, when 94 mg of weights 805 are used (compare to FIGS. 9C and 9D). As such, these images show that the presently disclosed suture 100 exhibits a considerably smaller bending radius than a prior-art suture such as the nylon suture that is comparably sized (i.e., USP 9-0) when tested under similar conditions (e.g., using the same apparatus 800 with the same weights 805).

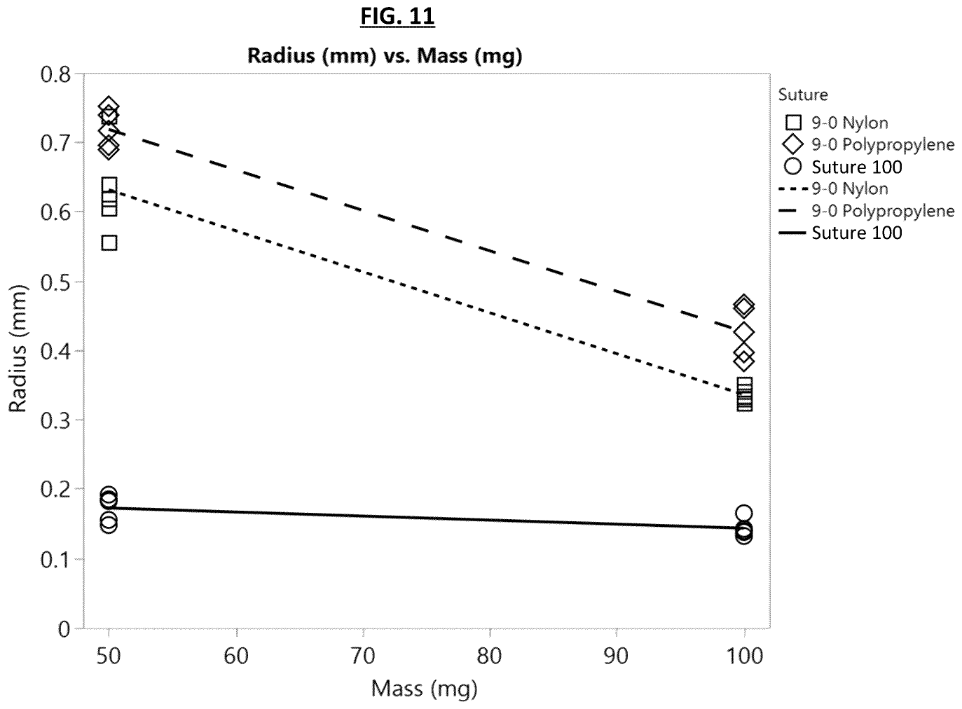

FIG. 11 shows a graph comparing the results of the bending stiffness tests performed on three different sutures (USP 9-0 nylon suture, USP 9-0 PP suture, and the suture 100 as disclosed herein that is comparable to a USP 9-0 suture) and two different weights (50 mg and 100 mg). Five measurements were made on different locations of each tested suture. Care was taken to select a location on the suture that had not been previously deformed during packaging or handling. The nylon suture is a black monofilament nylon suture sold under the ETHILON® trade name, part number 7717 (Ethicon, Inc; Raritan, NJ). The PP suture is a blue monofilament suture, sold under the PROLENE® trade name, part number W2780 (Ethicon, Inc.; Raritan, NJ). The suture 100 is the presently disclosed suture implementing the thread 102 shown in FIGS. 2, 3, 4, 5, and 6, as previously explained herein. The suture 100 is shown to consistently have a smaller bending radius than either of the comparably-sized (USP 9-0) nylon or PP suture, indicating a lower bending stiffness than either of these prior-art sutures.

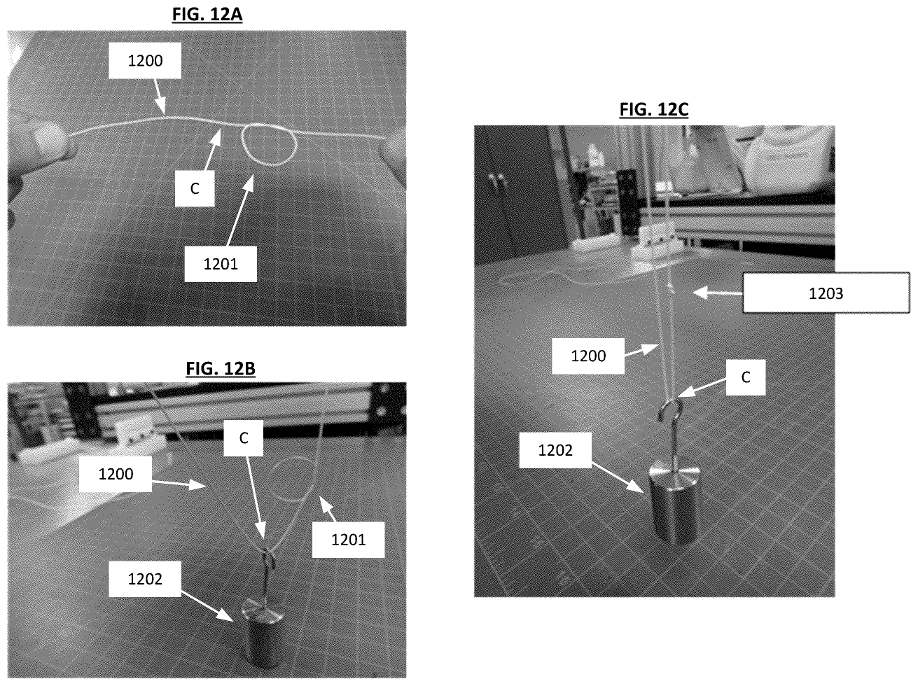

The “knot size” of the suture may be measured according to a method shown in FIGS. 12A through 12C. In FIG. 12A, a sample suture 1200 that is to be tested is configured such that a loose overhand knot 1201 is formed at a location that is off-center, that is, slightly off to one side of the center (“C”) of the suture 1200. The two ends of the suture 1200 may be held manually or may be affixed to two surfaces for stability. In FIG. 12B, on a stable surface, a weight 1202 is placed at or near the center C of the suture 1200, showing the loose knot 1201 being located to one side of the suture 1200 with respect to the center C. Any suitable mass may be used for the weight 1202, including but not limited to a 10-gram weight as shown in the figures. Any suitable coupling means such as a hook may be used to place the weight 1202 at the determined location of the suture 1200.

In FIG. 12C, the ends of the suture 1200 are brought closer together and are gently lifted from the surface on which the weight 1202 is placed. The tensioning force applied by this movement causes the knot 1201 to tighten to form a tensioned knot 1203 that has a considerably smaller radius as compared to the pre-tensioning knot 1201. The tensioning force is approximately ½ of the weight 1202 under suspension. Thereafter, an image (e.g., an SEM image) of the tensioned knot 1203 is captured such that the size, or maximum thickness, of each tensioned knot can be measured for each sample of suture 1200.

FIG. 13 is an example of the image taken at 500× magnification of a tensioned knot 1300 formed in the suture 100 (or more specifically, the thread 102 thereof) according to embodiments disclosed herein, with a scale showing the length of 100 μm relative to the image (such that a distance between two consecutive vertical markers represents 10 μm). Indicated at the bottom of the image is “9-0 blue ePTFE 3.00 kV X500” showing that the suture 100 or thread 102 is comparable to a USP 9-0 suture and includes ePTFE as its material. The knot size (labeled as such in the figure) is measured as a maximum thickness of the tensioned knot 1300 that is formed, and the maximum thickness may be measured along an axis that is substantially perpendicular to a longitudinal axis (or fiber axis) of the suture 100. As such, the knot size as measured in FIG. 13 is approximately 60 μm, for example. In some examples, the knot size may be defined by two dimensions: a length in a longitudinal direction (L1) and a length in an axial direction (L2) that is transverse to the longitudinal direction. The lengths L1 and L2 are both measured from one end of the knot to the opposite end of the knot. The tighter or more compressed the knot is, the smaller the difference would be between the first length L1 and the second length L2.

In some examples, the suture 100 or thread 102 may be a synthetic fiber having an axial length suitable for tissue ligation or approximation and a variable cross-section disposed along a portion of the axial length. In some examples, the variable cross-section may vary between an uncompressed state 1302 presenting a first cross-section defining a first peripheral edge circumscribing the fiber or thread 102 at the first cross-section, and a compressed state 1304 presenting a second cross-section defining a lesser second peripheral edge circumscribing the fiber or thread 102 at the second cross-section. The compressed state of portion 1304 may be present in the fiber or thread 102 within the knot 1300 formed by the fiber or thread 102, and the uncompressed state 1302 may be present in the fiber or thread 102 adjacent to or near the knot 1300.

In some examples, the edge-to-edge distance (e.g., the maximal cross-sectional dimension) of the fiber or thread 102 may be different between the unknotted state (D1) and the knotted state (D2). For example, when the knot is formed as shown, the edge-to-edge distance of the fiber or thread 102 at the knot (shown as D2) may be greater than the edge-to-edge distance of the fiber or thread 102 outside the knot (shown as D1). This difference may be facilitated by the presence of fibrils that are interconnected with respect to each other inside the fiber or thread 102, such that when a force is applied transversely with respect to the longitudinal axis of the fiber or thread 102, such as when forming a knot, the force causes the fibrils to be more dispersed than before, thereby causing an increase in the edge-to-edge distance at the knot. Along similar lines, the loose or distributed portions 701 previously referenced may assist with compression and flattening of the thread at the knotted region. As an illustrative example, FIG. 13 shows that the edge-to-edge distance of the fiber or thread 102 in the unknotted state (D1) is about 30 μm, and the edge-to-edge distance of the fiber or thread 102 in the knotted state (D2) is about 40 μm. As such, there is an increase of about 33% in the edge-to-edge distance of the fiber or thread 102 from the unknotted state to the knotted state.

In some examples, an uncompressed state 1302 may present a first cross-section having first openings disposed within the fiber or thread 102 at the first cross-section, and a compressed state 1304 may present a second cross-section having second openings disposed within the fiber at the second cross-section. The second openings may have a reduced size compared to the first openings. As referred to herein, the first and second openings refer to any of the pore openings 114 shown in FIGS. 6 and 7, for example, where the size of the openings 114 may increase or decrease accordingly as explained above.

FIGS. 13A and 13B are examples of the images taken at 500 magnification of a tensioned knot formed in prior-art ETHILON® nylon sutures according to the aforementioned test method, with a scale showing the length of 100 μm relative to each image (such that a distance between two consecutive vertical markers represents 10 μm). Indicated at the bottom of FIG. 13A is “9-0 nylon 3.00 kV X500” showing that the suture is a USP 9-0 nylon suture. Indicated at the bottom of FIG. 13B is “9-0 polypro 3.00 kV X500” showing that the suture is a USP 9-0 PP suture. The measured knot size of the USP 9-0 ETHILON® nylon suture of FIG. 13A is about 110 μm. The measured knot size of the USP 9-0 ETHILON® PP suture of FIG. 13B is about 160 μm. Furthermore, as shown in FIG. 13A, the edge-to-edge distance (or alternatively referred to as a cross-sectional dimension or a maximal cross-sectional dimension) of the USP 9-0 ETHILON® nylon suture in the unknotted state (D1) is about 35 μm, and the edge-to-edge distance of the USP 9-0 ETHILON® nylon suture in the knotted state (D2) is about 45 μm. As such, there is an increase of about 29% in the edge-to-edge distance of the USP 9-0 ETHILON® nylon suture from the unknotted state to the knotted state. Also, as shown in FIG. 13B, the edge-to-edge distance of the USP 9-0 ETHILON® PP suture in the unknotted state (D1) is about 37 μm, and the edge-to-edge distance of the USP 9-0 ETHILON® PP suture in the knotted state (D2) is also about 47 μm. As such, there is an increase of about 27% in the edge-to-edge distance of the USP 9-0 ETHILON® PP suture from the unknotted state to the knotted state.

The coefficient of friction of the suture may be measured using any suitable high-precision friction tester as known in the art, for example the Tetra Basalt-N2 Precision testing machine that is manufactured by Falex Corporation.

FIG. 14 is an enlarged view of a portion of FIG. 13 to show certain details such as microfilaments 1400 which may collectively or altogether form the synthetic fiber having an axial length suitable for tissue ligation or approximation, for example. In some examples, the synthetic fiber may be formed using one (monofilament) or more (multifilament) of the microfilaments as shown. The image shows three examples of microfilaments, including the layered 1400A, interconnected 1400B, and clumped 1400C. The layered microfilaments 1400A are shown in dotted lines, where each microfilament may have opposing microfilament ends. The ends of some of the microfilaments may engage each other end-to-end along a portion of the axial length of the synthetic fiber (which in the image extends from left to right). Only a portion of the end-to-end engaging microfilaments are shown for simplicity, but it is understood that the end-to-end engagement of the microfilaments may extend along a portion or an entirety of the axial length of the synthetic fiber, and in some examples, a portion of the plurality of microfilaments are disposed so that the opposing microfilament ends engage each other end-to-end to define a monofilament composed of engaged microfilaments extending at least a portion of the axial length of the fiber. The microfilaments may be separated by spaces to define a porous surface.

Also shown in FIG. 14 are interconnected microfilaments 1400B which may be interconnected in the sense that multiple monofilaments may be twisted about each other for at least a portion of the axial length, where the twisting may cause the monofilaments to be interconnected, intertwined, interweaved, intertwisted, and/or interlaced, for example, as shown by the twisted configuration of the dotted lines of the microfilaments 1400B. In some examples, the multiple monofilaments of the interconnected microfilaments 1400B may each define pathways along the axial length to define a braided structure formed by the multiple monofilaments (such as a first monofilament and a second monofilament, or more, as suitable) extending along at least a portion of the axial length. In some examples, the clumped microfilaments 1400C may be the result of any one or more of the above configurations of multiple microfilaments engaging with one another.

Any suitable biocompatible material for the suture, discussed herein, may be used in manufacturing the suture with the properties as described herein. In certain instances, the suture material may include a fluoropolymer, such as a polytetrafluoroethylene (PTFE) polymer or an expanded polytetrafluoroethylene (ePTFE) polymer. In some instances, the suture material may include, but are not limited to, a polyester, a silicone, a urethane, a polyethylene terephthalate, or another biocompatible polymer, or combinations thereof. In some instances, bioresorbable or bioabsorbable materials may be used, for example a bioresorbable or bioabsorbable polymer. In some instances, the suture material can include Dacron, polyolefins, carboxy methylcellulose fabrics, polyurethanes, or other woven, non-woven, or film elastomers.

In each of FIGS. 4, 7, 14, 15B, 15E, 15H, 16B, and 16G, a dotted square is added into the image to provide a consistent reference point among the different images for comparison, where the reference point is a square that measures 20 μm by 20 μm (hereinafter referred to as the “reference square”) to explain the surface texture and/or topography of the thread 102.

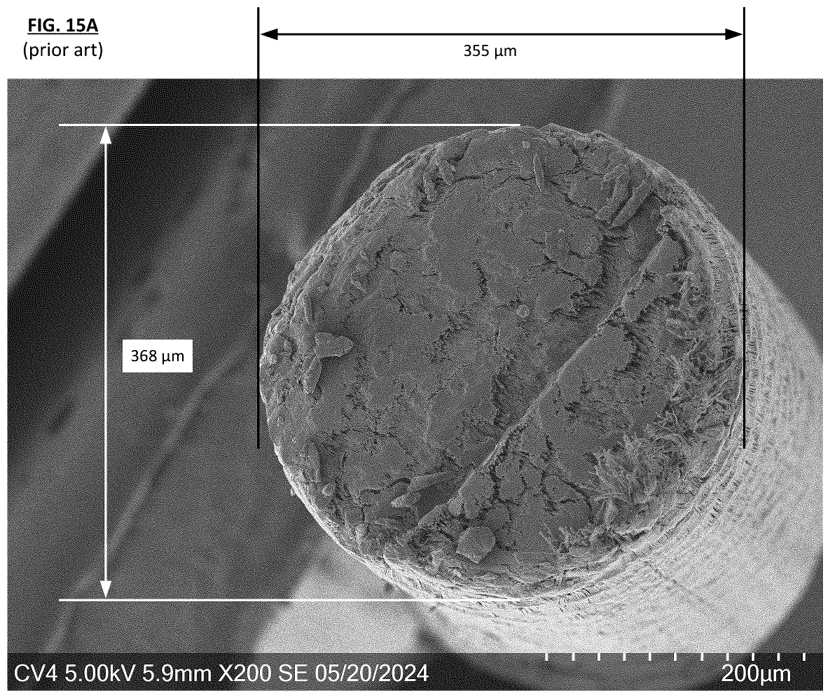

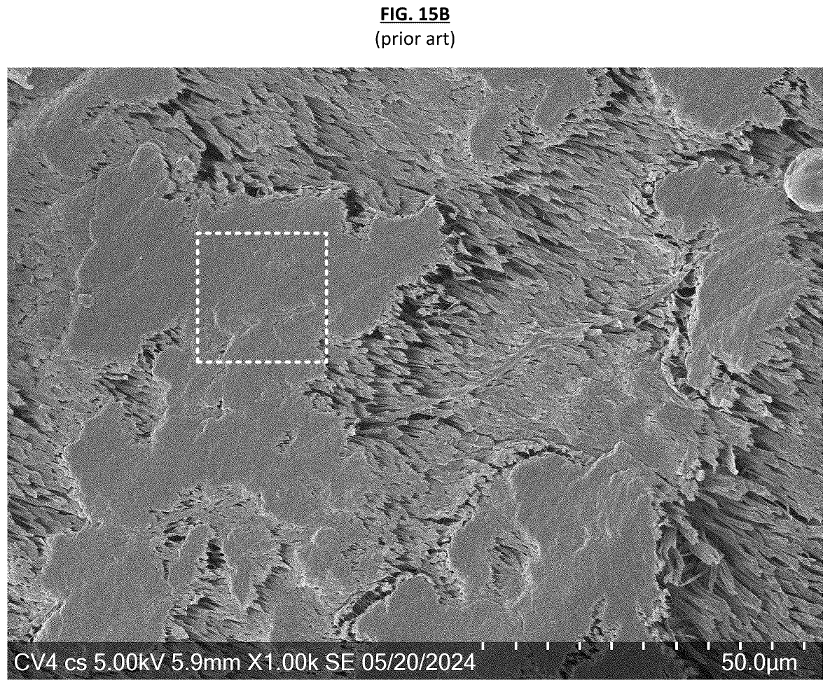

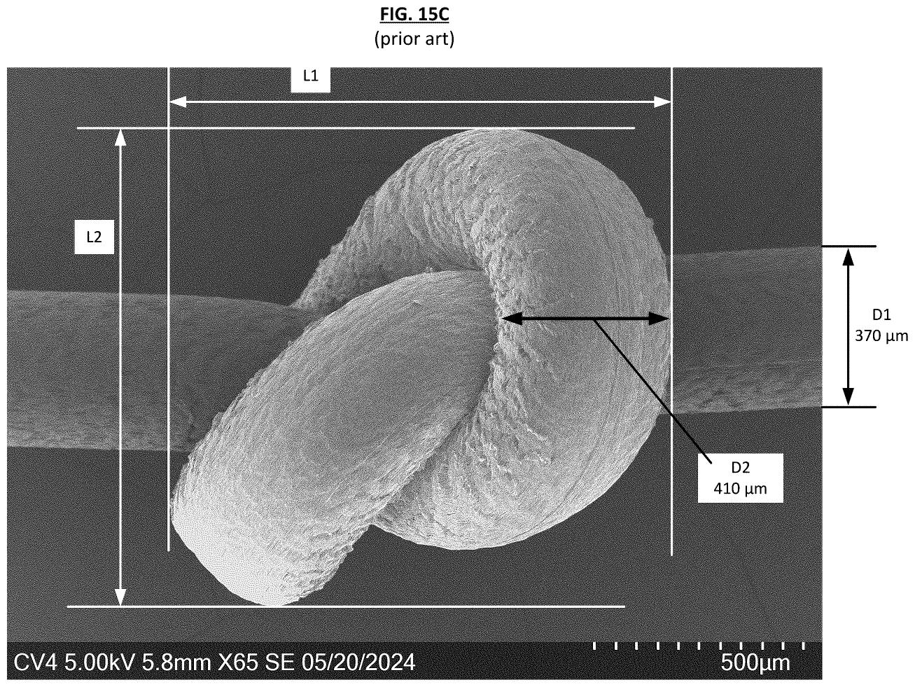

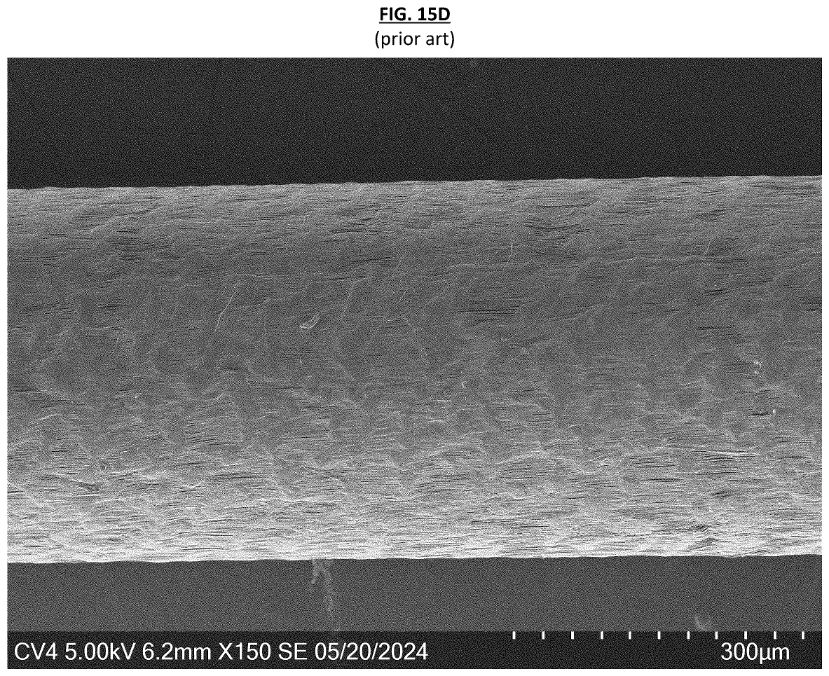

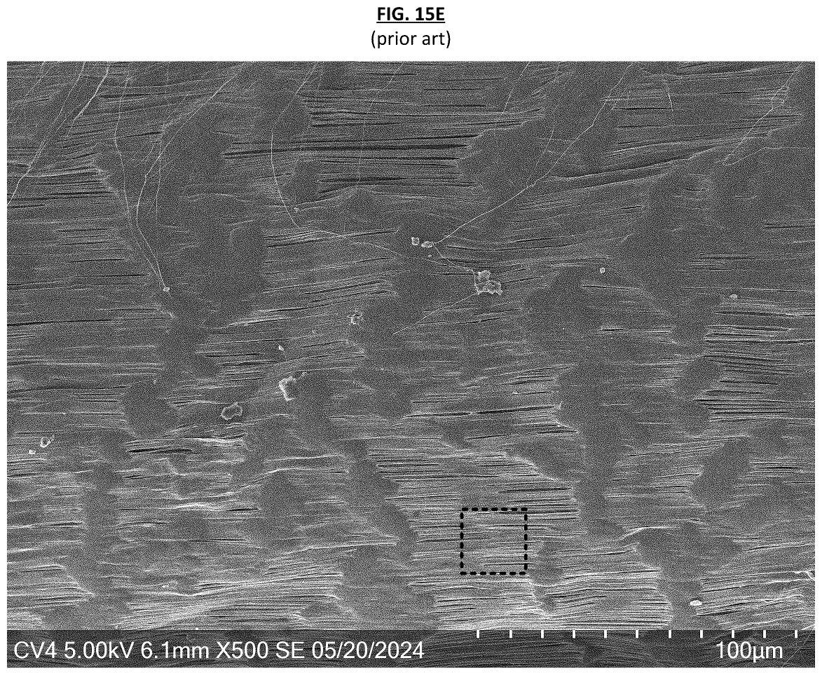

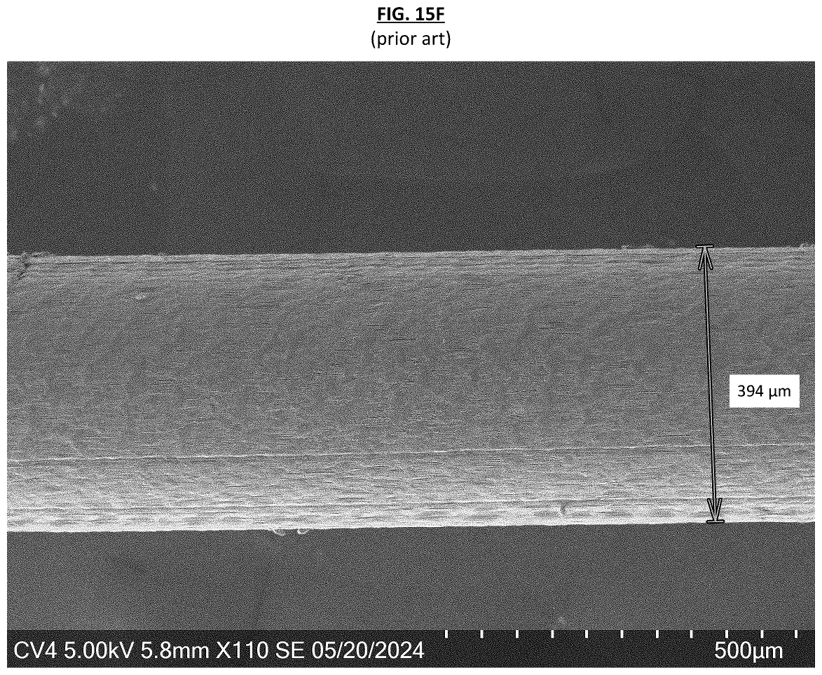

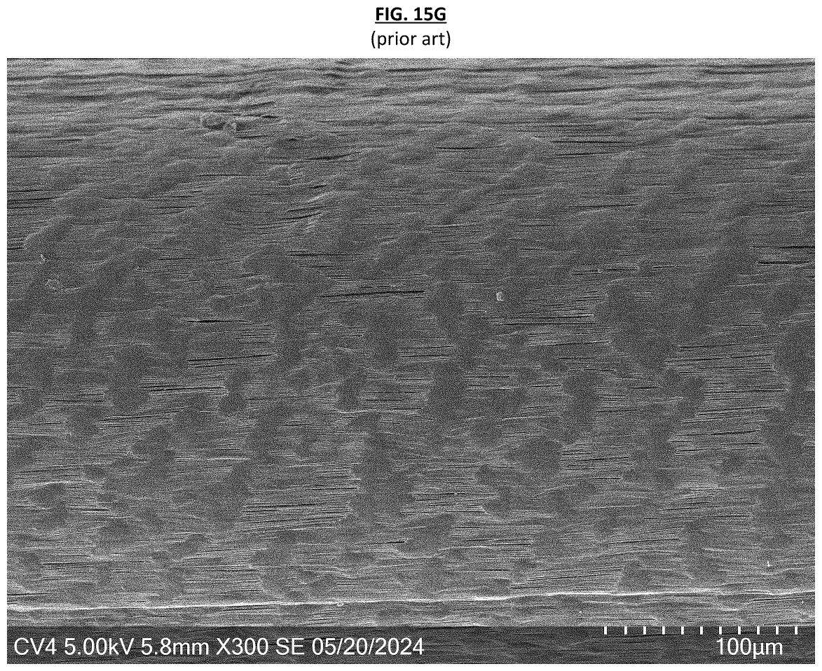

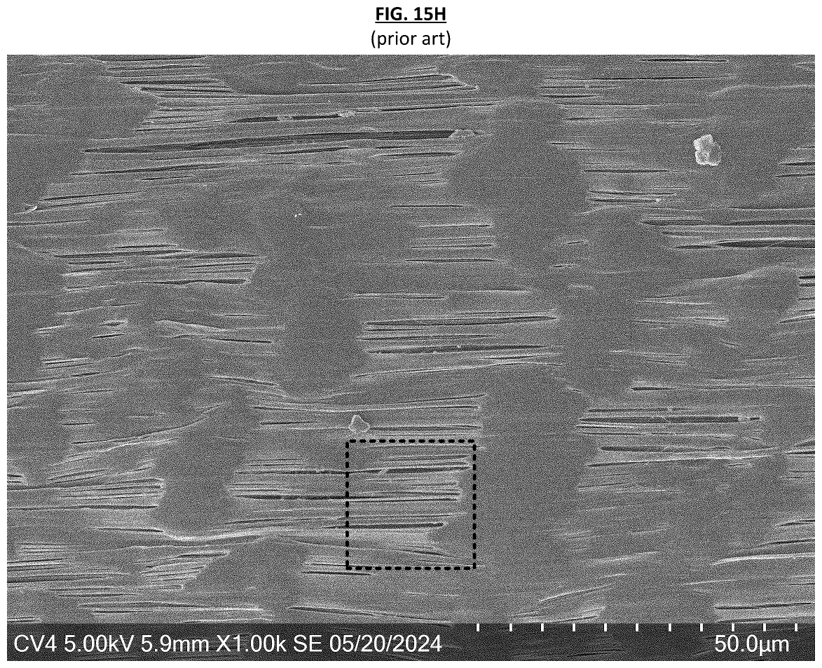

FIGS. 15A through 15H show images of a “CV4” suture as manufactured by W. L. Gore and Associates, Inc. in different configurations. For example, FIG. 15A shows a cross-sectional surface of the CV4 suture in which two edge-to-edge or maximal cross-sectional dimensions are measured, showing the dimensions of 355 μm by 368 μm. As such, the difference between the two dimensions is less than 5%, which makes the cross-sectional surface of the CV4 suture to be sufficiently round or circular. At the bottom of FIG. 15A is written “CV4 5.00 kV 5.9 mm X200 SE May 20, 2024” and a scale of 200 μm is shown such that the distance between each pair of neighboring lines is 20 μm.

FIG. 15B shows the details of a portion of FIG. 15A, and at the bottom of FIG. 15B is written “CV4 cs 5.00 kV 5.9 mm X1.00k SE May 20, 2024” and a scale of 50.0 μm is shown such that the distance between each pair of neighboring lines is 5 μm.

Shown in FIG. 15C is a knot that is formed using the CV4 suture, with the knot defined by the longitudinal length L1 (which is about 1180 μm) and the axial length L2 (which is about 1130 μm). The edge-to-edge distance of the CV4 suture in the unknotted state (D1) is 370 μm, and the edge-to-edge distance of the CV4 suture in the knotted state (D2) is 410 μm. As such, there is an increase of about 11% in the edge-to-edge distance of the CV4 suture from the unknotted state to the knotted state, which is considerably less than the increase of 33% as shown in FIG. 13, for example. At the bottom of FIG. 15C is written “CV4 5.00 kV 5.8 mm X65 SE May 20, 2024” and a scale of 500 μm is shown such that the distance between each pair of neighboring lines is 50 μm. This reduced thickening of the suture through the knot may be a result of having a relatively denser, or less compactible, structure at the interior of the cross-section (see FIG. 15A) in comparison to FIG. 7.

FIG. 15D shows another example of the CV4 suture, and at the bottom of the figure is written “CV4 5.00 kV 6.2 mm X150 SE May 20, 2024” and a scale of 300 μm is shown such that the distance between each pair of neighboring lines is 30 μm. FIG. 15E shows a magnified view of a portion of FIG. 15D, and at the bottom of the figure is written “CV4 5.00 kV 6.1 mm X500 SE May 20, 2024” and a scale of 100 μm is shown such that the distance between each pair of neighboring lines is 10 μm. As shown, the surface of the CV4 suture has a relatively denser, or smoother appearance when contrasted to that shown in FIG. 13, for example.

FIG. 15F shows another example of the CV4 suture, and at the bottom of the figure is written “CV4 5.00 kV 5.8 mm X110 SE May 20, 2024” and a scale of 500 μm is shown such that the distance between each pair of neighboring lines is 50 μm. The edge-to-edge distance of the CV4 suture in the unknotted state is shown as 394 μm. FIG. 15G shows a magnified view of a portion of FIG. 15F, and at the bottom of the figure is written “CV4 5.00 kV 5.8 mm X300 SE May 20, 2024” and a scale of 100 μm is shown such that the distance between each pair of neighboring lines is 10 μm. FIG. 15H shows a magnified view of a portion of FIG. 15G, and at the bottom of the figure is written “CV4 5.00 kV 5.9 mm X1.00k SE May 20, 2024” and a scale of 50.0 μm is shown such that the distance between each pair of neighboring lines is 5 μm. Again, as shown, the surface of the CV4 suture has a relatively denser, or smoother appearance when contrasted to that shown in FIG. 13, for example.

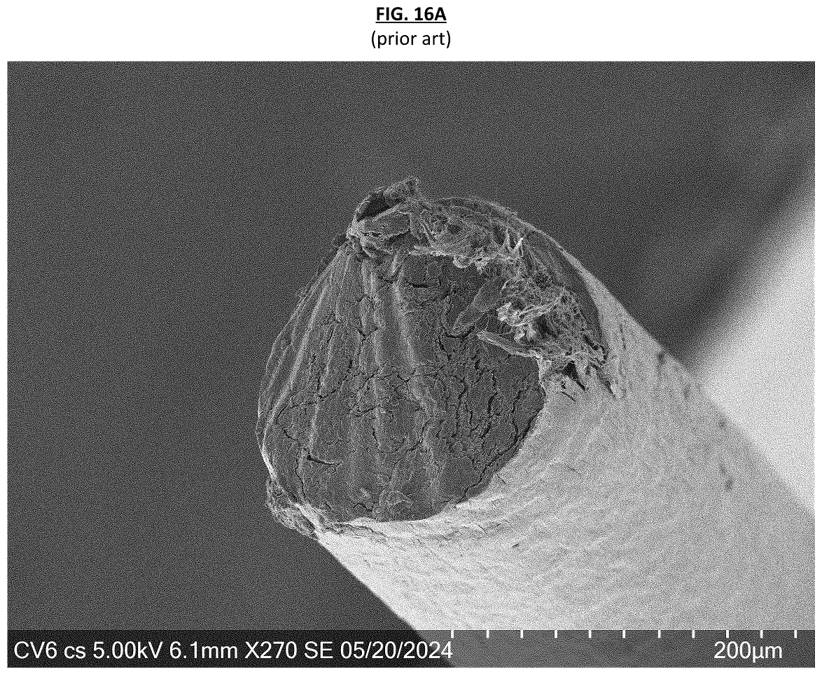

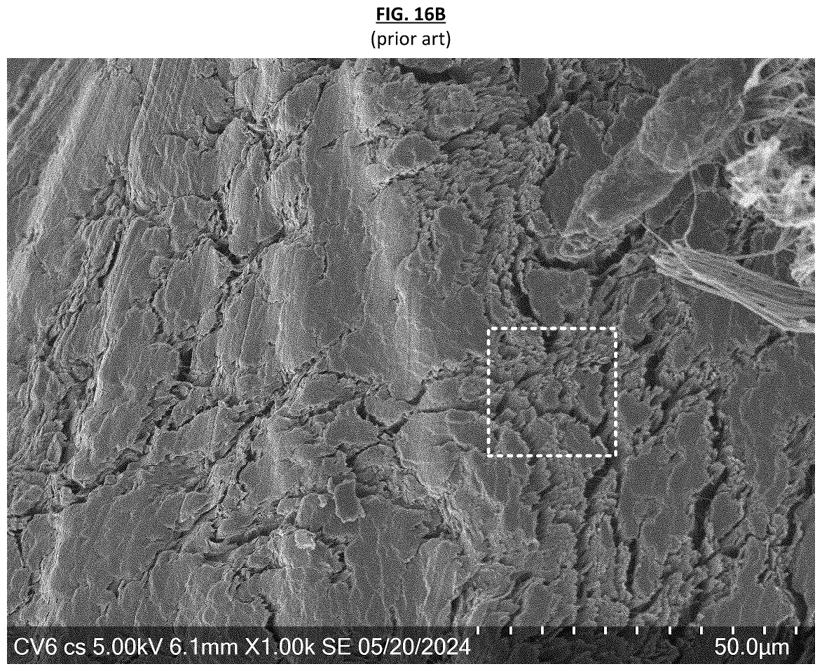

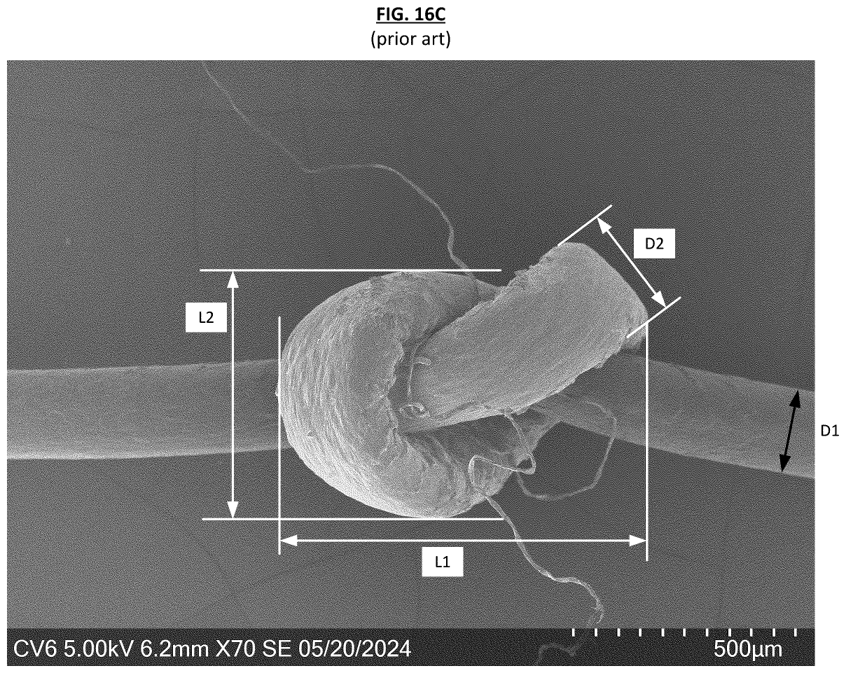

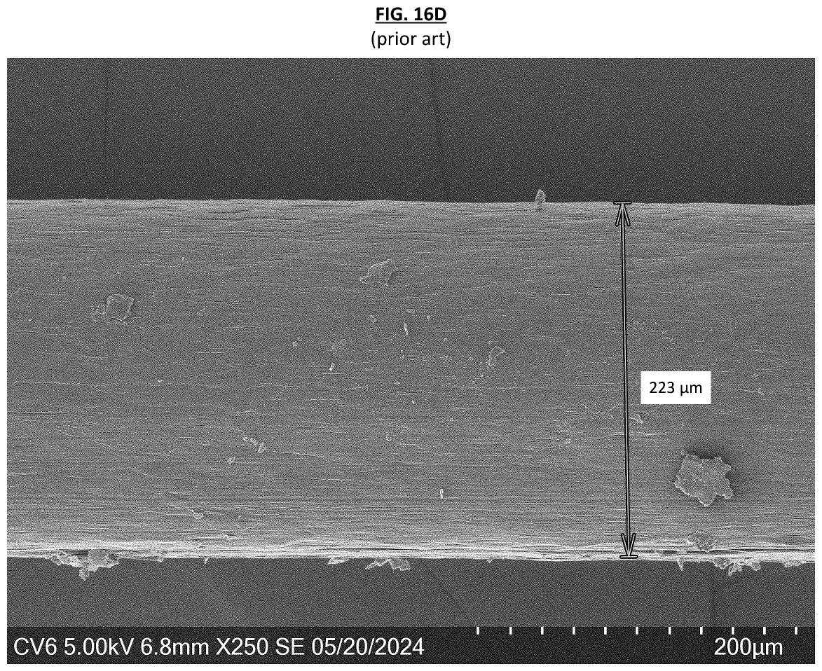

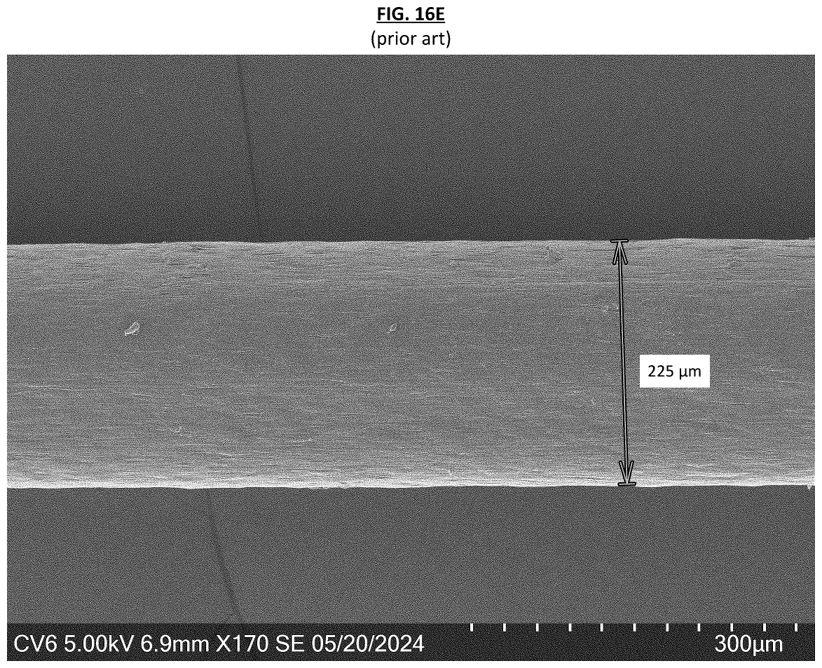

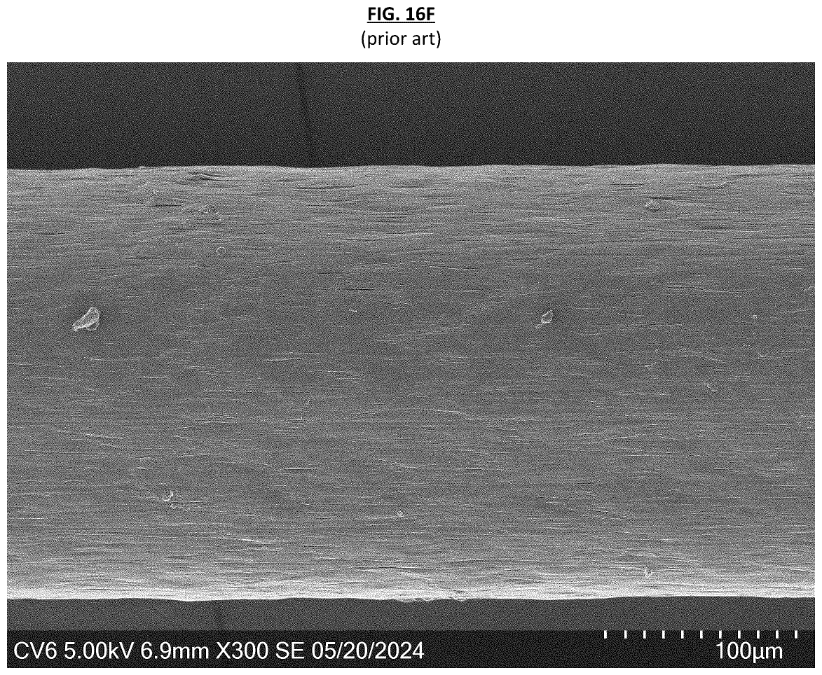

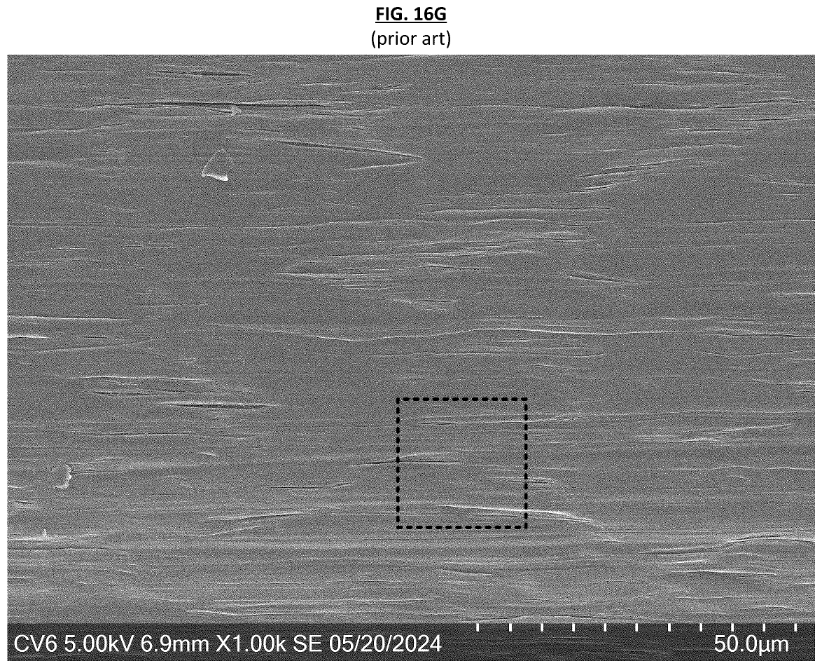

FIGS. 16A through 16G show images of a “CV6” suture as manufactured by W. L. Gore and Associates, Inc. in different configurations. For example, FIG. 16A shows a cross-sectional surface of the CV6 suture. At the bottom of FIG. 16A is written “CV6 cs 5.00 kV 6.1 mm X270 SE May 20, 2024” and a scale of 200 μm is shown such that the distance between each pair of neighboring lines is 20 μm. FIG. 16B shows the details of a portion of FIG. 16A, and at the bottom of FIG. 16B is written “CV6 cs 5.00 kV 6.1 mm X1.00k SE May 20, 2024” and a scale of 50.0 μm is shown such that the distance between each pair of neighboring lines is 5 μm. As shown, the surface of the CV6 suture also has a relatively denser, or smoother appearance when contrasted to that shown in FIG. 13, for example.

Shown in FIG. 16C is a knot that is formed using the CV6 suture, with the knot defined by the longitudinal length L1 (which is about 830 μm) and the axial length L2 (which is about 560 μm). The edge-to-edge distance of the CV6 suture in the unknotted state (D1) is about 190 μm, and the edge-to-edge distance of the CV6 suture in the knotted state (D2) is about 255 μm. As such, there is an increase of about 34% in the edge-to-edge distance of the CV6 suture from the unknotted state to the knotted state. At the bottom of FIG. 16C is written “CV6 5.00 kV 6.2 mm X70 SE May 20, 2024” and a scale of 500 μm is shown such that the distance between each pair of neighboring lines is 50 μm. However, the formed knot in FIG. 16C is not as compressed as the knot 1300 formed in FIG. 13, as indicated by the difference between the longitudinal length L1 and the axial length L2, or a ratio of the longitudinal length L1 to the axial length L2. When the difference between L1 and L2 is smaller, or the ratio of L1 to L2 is closer to one, the knot is deemed to be in a compressed configuration. In some examples, the knot may be deemed to be in the compressed configuration when forming the knot causes a greater reduction in the edge-to-edge length of the thread 102 or suture 100. FIG. 16D shows the details of a portion of FIG. 16C, the edge-to-edge distance being 223 μm in the unknotted state, and at the bottom of FIG. 16D is written “CV6 5.00 kV 6.8 mm X250 SE May 20, 2024” and a scale of 200 μm is shown such that the distance between each pair of neighboring lines is 20 μm.

FIG. 16E shows another example of the CV6 suture, the edge-to-edge distance being 225 μm in the unknotted state, and at the bottom of FIG. 16E is written “CV6 5.00 kV 6.9 mm X170 SE May 20, 2024” and a scale of 300 μm is shown such that the distance between each pair of neighboring lines is 30 μm. FIG. 16F shows the details of a portion of FIG. 16E, and at the bottom of FIG. 16F is written “CV6 5.00 kV 6.9 mm X300 SE May 20, 2024” and a scale of 100 μm is shown such that the distance between each pair of neighboring lines is 10 μm. FIG. 16G shows the details of a portion of FIG. 16F, and at the bottom of FIG. 16G is written “CV6 5.00 kV 6.9 mm X1.00k SE May 20, 2024” and a scale of 50.0 μm is shown such that the distance between each pair of neighboring lines is 5 μm.

In FIG. 4, the outer periphery or outer surface of the thread 102 is shown to be substantially smooth, or lacking visible roughness on the surface at the indicated magnification. In FIG. 7, the thread 102 may include relatively large openings or pore openings on the inside, including one or more pore openings 114 internally that extend for about 20 μm as measured along the maximal cross-sectional dimension 110, or up to about 38% of the maximal cross-sectional dimension 110 (which is about 53 μm in FIG. 7). In some examples, a total area occupied by the individual (discrete or unconnected) pore openings 114 that are detected inside the reference square may be from 10% to 20%, from 20% to 30%, from 30% to 40%, from 40% to 50%, or any other suitable value or range therebetween, with respect to the total area that is defined by both the pore openings 114 and the densified portions 700 (or, in some examples, the non-porous area) inside the reference square. The total area occupied by the individual (or unconnected) pore openings 114 may alternatively be referred to as the total area occupied by the loose portions 701 (or the porous area).

In FIG. 14, in some examples, the number of individual microfilaments 1400 that can be visually detected or observed on the surface of the thread 102 (for example, in a side view of the thread looking longitudinally, the surface of the thread would be the peripheral surface) inside the reference square may be from 15 to 20 microfilaments, from 20 to 40 microfilaments, from 40 to 60 microfilaments, from 60 to 80 microfilaments, from 80 to 100 microfilaments (or fibrils), or any other suitable value or range therebetween. In some examples, the number may be an average number that is calculated using a sample size of multiple (such as 5, 10, 15, 20, or more) measurements taken using the reference square. In some examples, only the fibrils that cross a perpendicularly bisecting line with respect to a longitudinal axis of the fiber or thread 102 are counted. In some examples, the microfilaments 1400 may be microfibrils that altogether form a monofilament. In some examples, the total number of microfilaments or fibrils that can be counted from one peripheral edge of the thread to the other opposing peripheral edge may be from 100 to 200, from 200 to 300, from 300 to 500, from 500 to 700, from 700 to 1000, or any other suitable value or range therebetween when viewed from a first angle, and the total number of microfilaments or fibrils that can be counted from one peripheral edge of the thread to the other opposing peripheral edge when viewed from the second angle that is perpendicular to the first angle may be from 1000 to 1200, from 1200 to 1500, from 1500 to 1700, from 1700 to 2000, or any other suitable value or range therebetween.

In comparison, for example referring to FIGS. 15B and 16B, the total area of pores on the cross-sectional surface (on average, within the reference square in the CV4 or CV6 suture) may be less than 10% the total area of the nonporous section on the surface that is visually observable in an SEM image provided at the indicated magnification. Furthermore, referring to FIGS. 15E, 15H, and 16G, the surface of the CV4 or CV6 suture may have less than 15 microfilaments that are visually observable as distinct structures within the reference square for an SEM image provided at the indicated magnification.

In some examples, the surgical suture 100 (or the thread 102 thereof) may be used in a medical device to engage with two or more components (first and second components) of the medical device, such as to secure one component (e.g., first component) to another component (e.g., second component). The suture 100 or thread 102 may also be used in coupling to a biocompatible component that is usable in a body of a patient, such as coupling the biocompatible component to the body of the patient. In some examples, the biocompatible component or the medical device may include at least one of (but are not limited to): an implant, a constraining sleeve, a graft, a valve leaflet, and/or a stent support frame, among others.

In some examples, a first component that is biocompatible and suitable for placement in a body of a patient and a second component that is biocompatible and suitable for placement in a body of a patient may be secured suing a securing method, and the method includes securing the first component to the second component using a surgical suture with a non-circular cross-section as disclosed herein. in some examples, the surgical suture has a transverse cross-section including densified areas surrounding openings disposed between the densified areas, and the method may further include forming a knot in the suture such that the synthetic fiber is externally compressed in a plane of the cross-section and the openings are collapsed, as disclosed herein. In some examples, the surgical suture has a transverse cross-section with a maximal cross-sectional dimension and a minimal cross-sectional dimension, and the method may further include forming a knot in the suture, where the knot includes a plurality of bends in the suture, and the suture is biased to orient with the maximal cross-sectional dimension perpendicular to the direction of curvature of at least one of the plurality of bends in the suture, as disclosed herein. According to some examples, the surgical suture may be a USP Class I nonabsorbable surgical suture sized to conform with at least one of USP size 12-0, USP size 11-0, USP size 10-0, USP size 9-0, and USP size 8-0, and optionally USP size 7-0 and USP size 6-0.

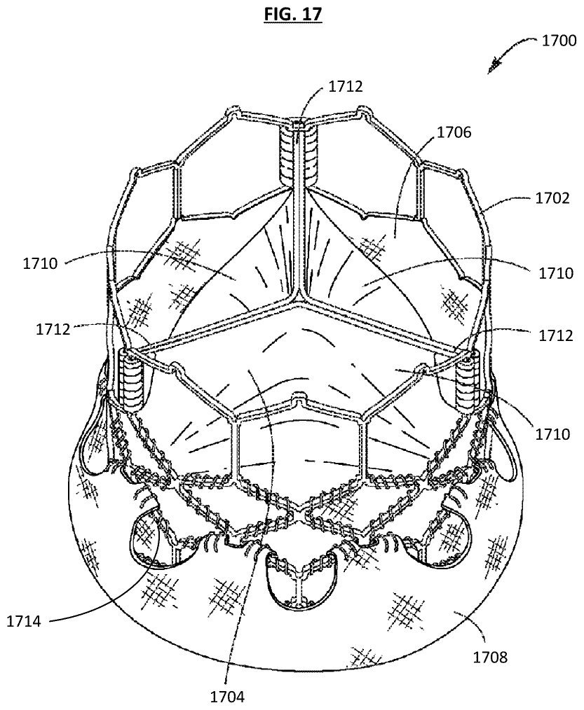

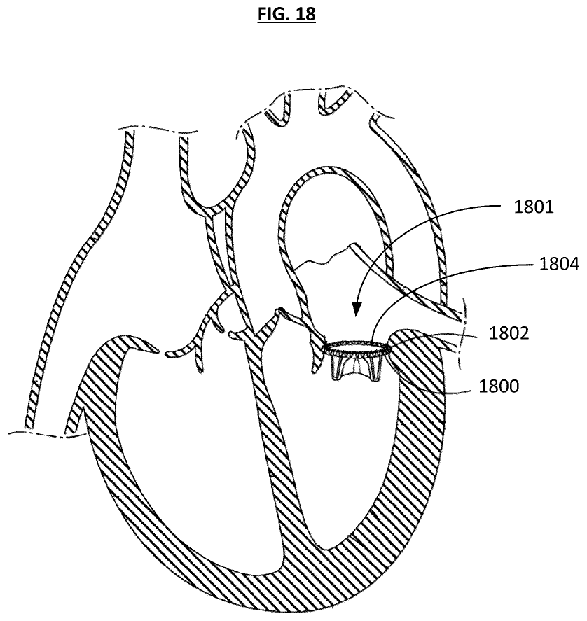

FIGS. 17 and 18 show examples of devices such as prosthetic heart valves in which the thread(s), fiber(s), or suture(s) as disclosed herein may be utilized or incorporated.

FIG. 17 shows an example of a prosthetic heart valve 1700 according to embodiments disclosed herein. The valve 1700 incorporates the features as disclosed in U.S. Pat. No. 9,393,110 B2, issued Jul. 19, 2016, to Edwards Lifesciences Corporation (Irvine, CA), further implementing the suture(s) or thread(s) as disclosed herein. The valve 1700 can have four main components: a stent or frame 1702, a valvular structure 1704, an inner skirt 1706, and an outer skirt 1708. The valvular structure 1704 can comprise three leaflets 1710, collectively forming a leaflet structure. The leaflets 1710 can be secured to one another at their adjacent sides to form commissures 1712 of the leaflet structure. The skirt 1706 can be secured to the inside of frame 1702 via sutures 1714 that can be appropriate-sized sutures selected from one or more of the embodiments described herein.

FIG. 18 shows an example of securing a prosthetic heart valve 1801 in a heart according to embodiments disclosed herein. The valve 1801 incorporates the features as disclosed in U.S. Pat. No. RE47,490 E, issued Jul. 9, 2019, to Edwards Lifesciences Corporation (Irvine, CA), further implementing the suture(s) or thread(s) as disclosed herein. The surgeon or other user may secure a prosthetic valve support frame to a native annulus of the heart via a sewing ring 1800 and/or other an attachment structure configured to be attached to, and/or to otherwise facilitate securement of the device to, the annulus of a native heart valve. For example, the surgeon or other user may run multiple sutures 1802 (e.g., 12-18 of such sutures) through the tissue of the native heart valve annulus and through the sewing ring 1800, and then tighten the sutures 1802 to secure the support frame 1804 at the native heart valve annulus.

In the aforementioned embodiments, the suture 100 or thread 102 as disclosed herein may be implemented as the sutures 1714 in FIG. 17 and the sutures 1802 in FIG. 18, for example.

The disclosure of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this disclosure provided they come within the scope of the appended claims and their equivalents.

What is claimed is:

1. A United States Pharmacopeia (USP) Class I nonabsorbable surgical suture comprising:
a synthetic fiber having an axial length suitable for tissue ligation or approximation, the synthetic fiber further having a non-circular cross-section maintained along at least a portion of the axial length of the synthetic fiber.

2. The USP Class I nonabsorbable surgical suture of claim 1, wherein the synthetic fiber has a cross-section disposed orthogonally to the length at a portion of the axial length, the cross-section including densified areas surrounding openings disposed between the densified areas, the openings being collapsible when the synthetic fiber is externally compressed in a plane of the cross-section.

3. The USP Class I nonabsorbable surgical suture of claim 1, wherein the synthetic fiber having side surfaces each extending axially along at least a portion of the axial length of the synthetic fiber, the axially-extending side surfaces including a flat surface disposed between two curved surfaces, each of the two curved surfaces curving about a fiber axis from 90 degrees to 180 degrees.

4. The USP Class I nonabsorbable surgical suture of claim 1, wherein the synthetic fiber has a plurality of microfilaments each having microfilament ends that engage each other end-to-end along a portion of the axial length of the synthetic fiber.

5. The USP Class I nonabsorbable surgical suture of claim 1, wherein the synthetic fiber has a cross-section disposed orthogonally to the length at a portion of the axial length, the cross-section being deformable from a first state to a second state, the first state presenting a first cross-sectional dimension that deforms to a lesser second cross-sectional dimension in the second state, the deforming being due to a reduction of openings disposed within the cross-section.

6. The USP Class I nonabsorbable surgical suture of claim 1, wherein the synthetic fiber having a cross-section disposed orthogonally to a fiber axis along a portion of the axial length, the cross-section defining a flat surface of the synthetic fiber extending along the portion of the axial length, the synthetic fiber predisposed to twist about the axis to present the flat surface as an inner surface of a curvature formed when the fiber is deformed to form the curvature.

7. The USP Class I nonabsorbable surgical suture of claim 1, wherein the synthetic fiber having a variable cross-section disposed along a portion of the axial length, the variable cross-section varying between an uncompressed state presenting a first cross-section defining a first peripheral edge circumscribing the fiber at the first cross-section and a compressed state presenting a second cross-section defining a lesser second peripheral edge circumscribing the fiber at the second cross-section, the compressed state being present in the fiber within a knot formed by the fiber, the uncompressed state being present in the fiber adjacent to the knot.

8. The USP Class I nonabsorbable surgical suture of claim 1, wherein the synthetic fiber having a variable cross-section disposed along a portion of the axial length, the variable cross-section varying between an uncompressed state presenting a first cross-section having first openings disposed within the fiber at the first cross-section and a compressed state presenting a second cross-section having second openings disposed within the fiber at the second cross-section, the second openings having a reduced size compared to the first openings, the compressed state being present in the fiber within a knot formed by the fiber, the uncompressed state being present in the fiber adjacent to the knot.

9. The USP Class I nonabsorbable surgical suture of claim 1, wherein the synthetic fiber includes a plurality of microfilaments with each microfilament having opposing microfilament ends, a portion of the plurality of microfilaments disposed so that opposing microfilament ends engage each other end-to-end to define a monofilament composed of engaged microfilaments extending at least a portion of the axial length.

10. The USP Class I nonabsorbable surgical suture of claim 1, wherein the synthetic fiber includes a plurality of microfilaments with each microfilament having opposing microfilament ends, a first portion of the plurality of microfilaments disposed so that opposing microfilament ends engage each other end-to-end to define a first monofilament composed of engaged microfilaments, a second portion of the plurality of microfilaments disposed so that opposing microfilament ends engage each other end-to-end to define a second monofilament composed of engaged microfilaments, the first and second monofilaments twisted about each other for at least a portion of the axial length.

11. The USP Class I nonabsorbable surgical suture of claim 1, wherein the synthetic fiber includes a plurality of microfilaments with each microfilament having opposing microfilament ends, a first portion of the plurality of microfilaments disposed so that opposing microfilament ends engage each other end-to-end to define a first monofilament composed of engaged microfilaments, a second portion of the plurality of microfilaments disposed so that opposing microfilament ends engage each other end-to-end to define a second monofilament composed of engaged microfilaments, the first and second monofilaments each defining pathways along the axial length to define a braided structure formed by the first and second monofilaments extending along at least a portion of the axial length.

12. A surgical suture comprising:
a synthetic fiber having an axial length suitable for tissue ligation or approximation, the synthetic fiber further defining a cross-section along at least a portion of the axial length of the synthetic fiber having a major dimension and a minor dimension, wherein the cross-section has a perimeter defined by a porous surface.

13. The surgical suture of claim 12, wherein the porous surface is characterized by a plurality of fibers of material separated by spaces.

14. The surgical suture of claim 13, wherein the plurality of fibers include a plurality of microfilaments with each microfilament having opposing microfilament ends, a portion of the plurality of microfilaments disposed so that opposing microfilament ends engage each other end-to-end to define a monofilament composed of engaged microfilaments extending at least a portion of the axial length.

15. The surgical suture of claim 13, wherein the plurality of fibers include a plurality of microfilaments with each microfilament having opposing microfilament ends, a first portion of the plurality of microfilaments disposed so that opposing microfilament ends engage each other end-to-end to define a first monofilament composed of engaged microfilaments, a second portion of the plurality of microfilaments disposed so that opposing microfilament ends engage each other end-to-end to define a second monofilament composed of engaged microfilaments, the first and second monofilaments twisted about each other for at least a portion of the axial length.

16. The surgical suture of claim 13, wherein the plurality of fibers include a plurality of microfilaments with each microfilament having opposing microfilament ends, a first portion of the plurality of microfilaments disposed so that opposing microfilament ends engage each other end-to-end to define a first monofilament composed of engaged microfilaments, a second portion of the plurality of microfilaments disposed so that opposing microfilament ends engage each other end-to-end to define a second monofilament composed of engaged microfilaments, the first and second monofilaments each defining pathways along the axial length to define a braided structure formed by the first and second monofilaments extending along at least a portion of the axial length.

17. A suture comprising:
a fiber formed of a synthetic, porous material, the fiber having a compressible, non-circular cross-section and defining a fiber axis and having an axial length suitable for tissue ligation or approximation and side surfaces each extending axially along at least a portion of the axial length of the synthetic fiber, the fiber being in a knotted configuration such that the fiber has a first portion defining a first porosity and a second portion defining a second porosity greater than the first porosity, the first portion corresponding to a knotted portion and the second portion corresponding to an unknotted portion.

18. The suture of claim 17, wherein the first porosity is defined by first pore openings, and the second porosity is defined by second pore openings having larger average pore sizes than the first pore openings.

19. The suture of claim 17, wherein the knotted portion has a knot size of from 25 μm to 100 μm.

20. A method of treating an eye condition, the method comprising:
using at least one suture for an ophthalmic ligation or approximation procedure, wherein the at least one suture comprises a synthetic fiber having an axial length suitable for tissue ligation or approximation, the synthetic fiber further having a non-circular cross-section maintained along at least a portion of the axial length of the synthetic fiber.
